(19) United States
(12) Patent Application Publication
(10) Pub. No.: US 2004/0086514 A1
(43) Pub. Date: May 6, 2004

(54) Novel helicobacter pylori-binding substances and use thereof

(76) Inventors: Karl-Anders Karlsson, Goteborg (SE); Irene Leonardsson, Ljungskile (SE); Susann Teneberg, Hindas (SE); Jonas Angstrom, Goteborg (SE)

Correspondence Address: BIRCH STEWART KOLASCH & BIRCH, PO BOX 747, FALLS CHURCH, VA, 22040-0747, US

(21) Appl. No.: 10/149,608

(22) PCT Filed: Dec. 15, 2000
(86) PCT No.: PCT/SE00/02567

(30) Foreign Application Priority Data
Dec. 15, 1999 (SE) 9904581-7

(51) Int. Cl. A61K 39/40; A61K 31/715; C07K 16/46

(52) U.S. Cl. 424/178.1; 514/54; 530/391.1; 536/123

(57) ABSTRACT

Helicobacter pylori-binding substances comprising Galβ3GlcNAc or Galβ3GalNAc are described, as well as use thereof in pharmaceutical compositions and food-stuff, and methods for treatment of conditions due to the presence of Helicobacter pylori. Also use of said substance for the identification of bacterial adhesions, for the production of a vaccine against Helicobacter pylori, for diagnosis of Helicobacter pylori infections, for typing of Helicobacter pylori, for identification of Helicobacter pylori binding substances and for inhibition of the binding of Helicobacter pylori is described.

### FIELD OF THE INVENTION

The present invention relates to novel Helicobacter pylori-binding substances and use thereof in e.g. pharmaceutical compositions and methods for treatment of conditions due to Helicobacter pylori.

### BACKGROUND OF THE INVENTION

Adhesion of microorganisms is regarded as a first step in pathogenesis of infections, where the specificity of the adhesins of the infectious agent on the one hand, and the receptor structures expressed by the epithelial cells of the host target organ on the other, are important determinants of the host range and the tissue tropism of the pathogen (1).

The human gastric pathogen Helicobacter pylori is an etiologic agent of chronic superficial gastritis (2), and has also been associated with the development of duodenal ulcer, gastric ulcer and gastric adenocarcinoma (3-7). This microorganism has a very distinct host range and tissue tropism, i. e. it requires the presence of human gastric-type epithelium for colonisation (8). In the human stomach most of the bacteria are found in the mucus layer, but selective association of the bacteria to surface mucous cells has also been shown (8, 9).

Several different binding specificities of Helicobacter pylori have previously been demonstrated. Thus the binding of the bacterium to such diverse compounds as phosphatidylethanolamine and gangliotetraosylceramide (10, 11), the Le^{b} blood group determinant (12), heparan sulphate (13), the GM3 ganglioside (14), sulfatide (14, 15), and lactosylceramide (16), has been reported. A sialic acid-dependent binding of Helicobacter pylori to large complex glycosphingolipids (polyglycosylceramides) of human erythrocytes, granulocytes and placenta has also been documented (17, 18).

Besides being associated with gastrointestinal diseases, Helicobacter pylori is associated with multiple diseases also affecting other organs than ones of gastrointestinal tract (74). For example associations with hearth diseases especially atherosclerosis (75), liver diseases including liver adenocarcinoma (76, 77), skin diseases (78), and sudden infant death syndrome (79, U.S. Pat. No. 6,083,756) have been indicated.

### SUMMARY OF THE INVENTION

The main object of the invention is to provide new ways to treat conditions caused by Helicobacter pylori.

The invention is based on the finding of a specific Helicobacter pylori receptor in the human gastric epithelium. The receptor is in many cases a glycolipid, lactotetraosylceramide, exclusively found in the human gastrointestinal tract, and during the research work it was shown that the minimum binding epitope is Galβ3GlcNAc or the very similar structure Galβ3GalNAc.

The invention thus relates to Helicobacter pylori-binding substances comprising said binding epitope, or analogues or derivatives thereof.

One object of the invention is to provide pharmaceutical compositions for treatment of conditions caused by Helicobacter pylori.

Another object of the invention is the use of the above mentioned Helicobacter pylori-binding substances for the production of pharmaceutical compositions for treatment of a condition due to the presence of Helicobacter pylori.

Another object of the invention is to provide a method for treatment of a condition due to the presence of Helicobacter pylori.

Another object of the invention is the use of the above mentioned Helicobacter pylori-binding substances for the identification of bacterial adhesins.

Another object of the invention is the use of the above mentioned Helicobacter pylori-binding substances for the inhibition of the binding of Helicobacter pylori for both therapeutical purposes and non-medical purposes such as in vitro assays.

Another object of the invention is the use of the above mentioned Helicobacter pylori-binding substances as lead compounds for the identification of other Helicobacter pylori-binding substances.

Another object of the invention is the use of the above mentioned Helicobacter pylori-binding substances in food-stuffs or as nutritional additives.

Another object of the invention is the use of the above mentioned Helicobacter pylori-binding substances or the above mentioned bacterial adhesins for the production of vaccines against Helicobacter pylori.

Another object of the invention is the use of the above mentioned Helicobacter pylori-binding substances in the diagnosis of Helicobacter pylori infections.

Yet another object of the invention is the use of the above mentioned Helicobacter pylori-binding substances in the typing of Helicobacter pylori.

### DETAILED DESCRIPTION OF THE INVENTION

As stated above the invention relates to a specific Helicobacter pylori-binding substance. In the work leading to the present invention a large array of different Helicobacter pylori strains were metabolically labelled with ³⁵S-methionine and examined for binding to a panel of different naturally occurring glycosphingolipids separated on thin-layer plates. Two distinct binding specificities were repeatedly detected by autoradiography. As previously described in detail Helicobacter pylori bind to lactosylceramide, gangliotriaosylceramide and gangliotetraosylceramide (16). The only binding activity initially detected in human material was to a compound in the tetraglycosylceramide region of the non-acid fraction from human meconium.

The glycosphingolipid composition of the human gastric epithelium is not well defined. However, in a recent study of glycosphingolipids of the mucosal cells and submucosal tissue of the human gastrointestinal tract (55), an enrichment of sulfatides in the fundic and antral mucosa of the stomach was reported. The major non-acid glycosphingolipids migrated as galactosylceramide, lactosylceramide, globotriaosylceramide and globoside on thinlayer plates, while the main gangliosides migrated as GM3, GM 1 and GD3. Helicobacter pylori-binding lactosylceramide with phytosphingosine and hydroxy fatty acids has also been characterised in the epithelial cells of human stomach (16).

In addition, the blood group Cad-active ganglioside (GalNAcβ4 (NeuAcα3) Galβ4GlcNAcβ3Galβ4Glcβ1Cer) has been identified in the fundus region of human stomach (56), whereas it was not found in the pyloric region (57), indicating a differential expression of glycosphingolipids in different regions of the human stomach.

Due to limited access to human gastric tissue, the inventors initially concentrated on the Helicobacter pylori-binding glycosphingolipid detected in human meconium, which is the first sterile faeces of the newborn and consists mainly of extruded mucosal cells from the developing gastrointestinal tract. After isolation, this Helicobacter pylori-binding glycosphingolipid was characterised by mass spectrometry, proton NMR spectroscopy and methylation analysis as Galβ3GlcNAcβ3Galβ4Glcβ1Cer (lactotetraosylceramide). The tissue distribution of lactotetraosylceramide is very limited. Until recently lactotetraosylceramide had only been identified in human meconium (45), in the small intestine of an individual previously resected ad modum Billroth II (46), in normal human gastric mucosa and in human gastric cancer tissue (58) However, the “normal” mucosa, in 4 of the 5 cases described in the latter report, was obtained by antrectomy due to duodenal or gastric ulcer. Immunohistochemical studies, using the monoclonal antibody K-21, demonstrated a selective expression of the Galβ3GlcNAc-sequence in superficial human gastric mucosa (foveolar epithelium) of non-secretor individuals (59), coinciding with the localisation of Helicobacter pylori-binding to tissue sections (8, 9). An immunohistochemical study, utilising polyclonal antibodies binding to the Galβ3GlcNac-sequence, showed the presence of lactotetraosylceramide in the brush border cells of human jejunum and ileum of blood group OLe(a−b−)non-secretor individuals, and also of one individual with the blood group OLe(a+b+)non-secretor (60).

Among the 66 Helicobacter pylori isolates analysed in this study, 57 strains (86%) were found to express the lactotetraosylceramide binding specificity, whereas 9 strains were negative. The high prevalence of the lactotetraosylceramide binding property observed among the Helicobacter pylori isolates demonstrates that it is a conserved property of this gastric pathogen, and may thus represent an important virulence factor.

The biological relevance of the lactotetraosylceramide binding specificity was further substantiated by the binding of Helicobacter pylori to the tetraglycosylceramide region of the non-acid glycosphingolipids isolated from the target epithelial cells of human stomach. By proton NMR spectroscopy, and gas chromatography—mass spectrometry of permethylated tetrasaccharides obtained by ceramide glycanase hydrolysis, it was demonstrated that the binding-active fraction contained lactotetraosylceramide. The binding-active lactotetraosylceramide was only found in one of seven individuals analysed, which is suggestive in view of the fact that although infection with Helicobacter pylori and the associated chronic gastritis are very common, but only a small fraction of those infected develops any further consequences such as peptic ulcer or gastric adenocarcinoma (7). A speculative theory is thus that the presence of lactotetraosylceramide on the gastric epithelial cells is one of the co-factors necessary for the development of severe consequences of the infection, as peptic ulcer disease or gastric cancer.

The binding-active lactotetraosylceramide fraction isolated from human meconium contained both hydroxy and non-hydroxy ceramide species. Theoretically, the binding could thus be confined to the species with hydroxy ceramides, as described for the lactosylceramide binding specificity (16). However, lactotetraosylceramide isolated from rabbit thymus, with a ceramide composed exclusively of sphingosine and non-hydroxy 16:0 and 24:0 fatty acids (B. Lanne et al., to be published), was as active as the lactotetraosylceramide from human meconium (not shown), demonstrating that the binding to lactotetraosylceramide was not dependent on the ceramide composition.

The binding pattern obtained with ¹²⁵I-labeled bacterial surface proteins were identical to those obtained with ³⁵S-labeled whole bacterial cells, suggesting that these surface protein preparations may be utilised for isolation and characterisation of the carbohydrate-binding adhesins.

In summary, the adherence of Helicobacter pylori to the mucosal cells of human stomach appears to be a multi-component system where several bacterial adhesins recognise and bind to different receptors in the target tissue. This study identifies yet another binding-active compound, i.e. lactotetraosylceramide, detected by binding to glycosphingolipids on thin-layer plates. The distribution of this glycosphingolipid is limited, and hitherto it has only been found in the human gastrointestinal tract. In other human tissues lactotetraosylceramide is substituted with fucose or sialic acid, and thereby non-binding under the assay conditions used.

The isolation and structural characterisation of this Helicobacter pylori-binding glycosphingolipid, and the identification of the same compound in human gastric mucosal cells, lead to the identification of a minimum binding epitope, namely Galβ3GlcNAc. The epitope Galβ3GalNAc is very similar, both structurally and functionally, to Galβ3GlcNAc, and they are thus substantially interchangeable.

The invention thus relates to Helicobacter pylori-binding substances comprising at least one compound having Formula 1:

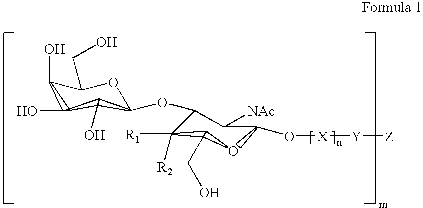

wherein:

R₁ and R₂ is H or OH, under the provision that when R₁ is H R₂ is OH, and when R₁ is OH R₂ is H;

X is a monosaccharide or oligosaccharide residue, and preferably X is lactosyl-, galactosyl-, poly-N-acetyl lactosaminyl, or forms part of an O-glycan or an N-glycan oligosaccharide sequence;

Y is nothing, a spacer group or a terminal conjugate, like a ceramide lipide moiety or linkage (—O—) to Z;

Z is an oligovalent or a polyvalent carrier or —H;

n is 0 or 1;

m is an integer equal to or larger than 1, and m may be up to several thousands or several millions depending on the substance,

or an analogue or derivative thereof having the same or better binding activity as the compound having formula I with regard to Helicobacter pylori.

When R₁ is OH and R₂ is H in Formula 1 the compound. with Formula 2 is obtained, and when R₁ is H and R₂ is OH the compound with Formula 3 is obtained.

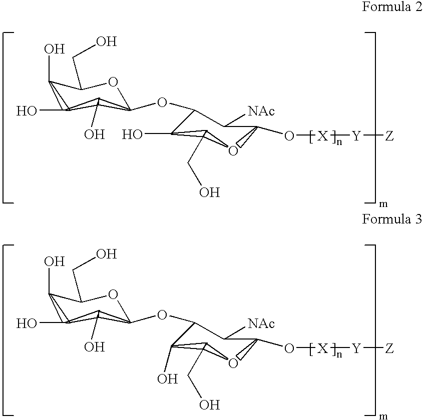

The invention also includes substances according to Formulas 1, 2 and 3, wherein —O—X is replaced by —S—X, N—X or —C—X, since man skilled in the art knows that these are interchangeable.

The invention also relates to Helicobacter pylori-binding substances comprising or consisting of Galβ3GlcNAc (corresponding to Formula 1 wherein R₁=OH and R₂=H) or Galβ3GalNAc (corresponding to Formula 1 wherein R₁=H and R₂=OH), or an analogue or derivative thereof having the same or better binding activity as Galβ3GlcNAc or Galβ3GalNAc with regard to Helicobacter pylori.

According to the invention it is possible to use Galβ3GlcNAc or Galβ3GalNAc per se, or any naturally occurring or synthetically produced analogue or derivative thereof having the same or better binding activity with regard to Helicobacter pylori. It is also possible to use a substance, such as lactotetraose, lactotetraosylceramide (Galβ3GlcNAcβ3Galβ4Glcβ1Cer) or gangliotetraosylceramide (Galβ3GalNAcβ4Galβ4Glcβ1Cer), comprising the binding site Galβ3GlcNAc or Galβ3GalNAc, or an analogue or derivative thereof having the same or better binding activity with regard to Helicobacter pylori. It may be preferable that said minimum binding epitope, or analogue or derivative thereof, is situated at a terminal non-reducing end of said substance.

It may be preferable to use lactotetraose or gangliotetraose, either alone or in a multivalent form.

The Helicobacter pylori-binding substance according to the invention may also consist of or comprise a carrier to which one or more of the above mentioned substances has/have been attached.

The Helicobacter pylori-binding substance according to the invention may also consist of or comprise a micelle comprising one or more of the above mentioned substances. One example of such a micelle is a liposome containing e.g. several lactotetraose molecules.

The Helicobacter pylori-binding substance according to the invention may also be conjugated to a polysaccharide, such as a polylactosamine chain or a conjugate thereof, or to an antibiotic, preferably an antibiotic with effect against Helicobacter pylori.

The substances according to the present invention may thus be part of a saccharide chain or glycoconjugate or mixture of glycocompounds containing other known Helicobacter binding epitopes, with different saccharide sequences and conformations, like Lewis b [Fucα2Galβ3(Fucα4)GlcNAc] or NeuNAcα3Galβ4Glc/GlcNAc. Using several binding substances together may be beneficial for therapy.

The substance according to the invention may be conjugated to an antibiotic substance, preferentially a penicillin type antibiotic. The substance according to the invention targets the antibiotic to Helicobacter pylori. Such conjugate is beneficial in treatment because lower amount of antibiotic is needed for treatment or therapy against Helicobacter pylori, which leads to lower side effect of the antibiotic. The antibiotic part of the conjugate is aimed to kill or weaken the bacteria, but the conjugate may also have an antiadhesive effect as described below.

It is known that Helicobacter pylori can bind several kinds of oligosaccharide sequences. Some of the binding by specific strains may represent more symbiotic interactions that do not lead to cancer or severe conditions. The present data about binding to cancer-type saccharide epitopes indicates that the substance according to the invention can prevent more pathologic interactions, in doing this it may leave some of the less pathogenic Helicobacter pylori bacteria/strains binding to other receptor structures. Therefore total removal of the bacteria may not be necessary for the prevention of the diseases related to Helicobacter pylori. The less pathogenic bacteria may even have a probiotic effect in prevention of more pathogenic strains of Helicobacter pylori.

It is also realised that Helicobacter pylori contains Galβ3GlcNAc-sequences on its surface which at least in some strains in non-fucosylated form which can be bound by the bacterium as described by the invention. The substance according to the invention can also prevent the binding between Helicobacter pylori bacteria and that way inhibit bacteria for example in process of colonisation.

The Helicobacter pylori-binding substance according to the invention may be e.g. a glycolipid, a glycoprotein or a neoglycoprotein. It may also be an oligomeric molecule comprising at least two oligosaccharide chains.

In order to treat a disease or a condition due to the presence of Helicobacter pylori in the gastrointestinal tract of a patient it is possible to use the substance according to the invention for anti-adhesion, i.e. to inhibit the binding of Helicobacter pylori to the receptors in the gastric epithelium of the patient. When the substance or pharmaceutical composition according to the invention is administered it will compete with the receptor in the binding of the bacteria, and all or some of the bacteria present in the gastrointestinal tract will then bind to the substance according to the invention instead of to the receptor on the gastric epithelium. The bacteria will then pass through the intestines and out of the patient attached to the substance according to the invention, resulting in a reduced effect of the bacteria on the patient's health. Preferably the substance used is a soluble compound comprising the binding site Galβ3GlcNAc or Galβ3GalNAc, such as soluble analogue of lactotetraose, lactotetraosylceramide, gangliotetraose or gangliotetraosylceramide. It is also possible, and often preferable, to attach the substance according to the invention to a suitable carrier. When a carrier is used several molecules of the substance according to the invention may be attached to one carrier, thus improving the inhibitory efficiency.

According to the invention it is also possible to treat other diseases due to the presence of Helicobacter pylori, such as liver diseases, heart diseases or sudden infant death syndrome.

According to the invention it is possible incorporate the substance according to the invention, optionally together with a carrier, in a pharmaceutical composition suitable for treatment of a condition due to the presence of Helicobacter pylori in the gastrointestinal tract of a patient or to use the substance according to the invention in a method for treatment of such a condition. Examples of conditions treatable according to the invention are chronic superficial gastritis, duodenal ulcer, gastric ulcer, and gastric adenocarcinoma.

The pharmaceutical composition according to the invention may also comprise other substances, such as an inert vehicle, or pharmaceutical acceptable adjuvants, carriers, preservatives etc., which are well known to persons skilled in the art.

Furthermore, the substance according to the present invention may be administered together with other drugs like drugs used to cure gastric diseases including proton pump inhibitors or gastric pH regulating drugs (omeprazole, lansoprazole, ranitidin etc.) and antibiotics used against Helicobacter pylori.

The substance or pharmaceutical composition according to the invention may be administered in any suitable way, although it is preferable to use oral administration.

The term “treatment” used herein relates to both treatment in order to cure or alleviate a disease or a condition, and to treatment in order to prevent the development of a disease or a condition. The treatment may either be performed in an acute or in a chronic way.

The term “patient”, as it is used herein, relates to any human or non-human mammal in need of treatment according to the invention. Furthermore, it is possible to use the substance according to the invention in order to identify one or more adhesins by screening for sequences that binds to the substance according to the invention. Said sequences may be, e.g., proteins or carbohydrates. The carbohydrate binding protein may be a lectin or a carbohydrate binding enzyme. The screening can be done for example by affinity chromatography or affinity cross linking methods.

Furthermore, it is possible to use substances specifically binding Galβ3GlcNAc or Galβ3GalNAc present on human tissues and thus prevent the binding of Helicobacter pylori. Examples of such substances include the monoclonal antibody K-21, specific for Galβ3GlcNAc and other antibodies or lectins binding the structure, or galactosidase enzyme capable of cleaving β3-linked galactoses or lacto-N-biosidase, endoglycosidase enzyme which releases terminal Galβ3GlcNAc from oligosaccharide chains. Moreover the adhesin binding Galβ3GlcNAc or especially the binding part of it may be used to inhibit the binding of Helicobacter pylori to the receptor Galβ3GlcNAc. When used in humans the binding substance should be suitable for such use such as a humanised antibody or a recombinant glycosidase of human origin that is non-immunogenic and capable of cleaving the terminal monosaccharide residue/residues from the substances of the invention. However, in gastrointestinal tract many naturally occurring lectins and glycosidases originating for example from food are tolerated.

Furthermore, it is possible to use the substance according to the invention as a template in order to produce a vaccine suitable for vaccination against Helicobacter pylori, such as the above mentioned conditions.

Furthermore, it is possible to use the substance according to the invention in the diagnosis of a condition due to a Helicobacter pylori infection.

Furthermore, it is possible to use the substance according to the invention for the inhibition of the binding of Helicobacter pylori for non-medical purposes, such as in an in vitro-assay system, which e.g. may be used for the identification of other Helicobacter pylori-binding substances.

Furthermore, it is possible to use the substance according to the invention as a lead compound in the identification of other Helicobacter pylori-binding substances.

Furthermore, it is also possible to use the substance according to the invention for typing of Helicobacter pylori.

Finally, it is also possible to use the substance according to the invention in a food-stuff, or in a nutritional composition, both for humans and animals, for example in food, milk, yoghurt, or other dairy product, beverage compositions and infant formula foods. The nutritional composition or food-stuff described here is not natural human milk. It is preferred to use the substance according to invention as a part of a so called functional or functionalised food. The said functional food has a positive effect on the health of the person or the animal by inhibiting or preventing the binding of Helicobacter pylori to target cells or tissues. The substance according to the invention can be a part of defined food or functional food composition. The functional food can contain other known food ingredients accepted by authorities controlling food like Food and Drug Administration in USA. The substance according to invention can be also used as nutritional additive, preferentially as a food or a beverage additive to produce a functional food or a functional beverage. The food or food additive can be also produced by having a cow or other animals to produce the substance according to invention in larger amounts naturally in its milk. This can be accomplished by having the animal over-express suitable glycosyltransferases in its milk. A specific strain or species of a domestic animal can be chosen and bread for larger production of the substance according to the invention. The substance according to the present invention and especially the substance according to invention for a nutritional composition or nutritional additive can be also produced by a micro-organism/s like a bacterium or yeast.

It is especially useful to have the substance according to the invention as part of a food-stuff or a nutritional composition for an infant or baby, preferentially as a part of an infant formula food. “Infant formula food” refers herein also to special infant formula foods like protein hydrolysed formula, formula for low-birth-weight infants or a follow-up formula. Many infants are fed by special formulas in replacement of natural human milk. The formulas may lack the special lactose based oligosaccharides of human milk especially the elongated ones like lacto-N-tetraose, Galβ3GlcNAcβ3Galβ4Glc, and its derivatives. The infant formula may be powder dried and it is reconstituted with water to give final food to be used by an infant or a baby. In a preferred embodiment the infant food is aimed for use having similar concentration of lacto-N-tetraose as present in natural human milk, about 0.05-5 g per litre, more preferentially 0.1-0.5 g per litre.

The lacto-N-neotetraose and para-lacto-N-hexaose Galβ3GlcNAcβ3Galβ4GlcNAcμ3Galβ4Glc are known from human milk and can be therefore considered as safe additives or ingredients in an infant food. Helicobacter pylori is especially infective with regard to infants or young children, and considering the diseases it may later cause it is reasonable to prevent the infection. Helicobacter pylori is also known to cause sudden infant death syndrome, but the strong antibiotic treatments used to eradicate the bacterium may be especially unsuitable for young children or infants.

When the substance according to the invention is to be used for diagnosis or typing, it may e.g. be included in e.g. a probe or on a test stick, optionally constituting part of a test kit. When this probe or test stick is brought into contact with a sample containing Helicobacter pylori, the bacteria will bind to the probe or test stick and can thus be removed from the sample and further analysed.

The glycosphingolipid nomenclature follows the recommendations by the IUPAC-IUB Commission on Biochemical Nomenclature (CBN for Lipids: Eur. J. Biochem. (1977) 79, 1121, J. Biol. Chem. (1982) 257, 3347-3351, and J. Biol. Chem. (1987) 262, 13-18).

It is assumed that Gal, Glc, GlcNAc, GalNAc, NeuAc and NeuGc are of the D-configuration, Fuc of the L-configuration, and all sugars present in the pyranose form.

Furthermore, lactotetraose, Galβ3GlcNAcβ3Galβ4Glc, is also known as lacto-N-tetraose.

In the shorthand nomenclature for fatty acids and bases, the number before the colon refers to the carbon chain length and the number after the colon gives the total number of double bonds in the molecule. Fatty acids with a 2-hydroxy group are denoted by the prefix h before the abbreviation e.g. h16:0. For long chain bases, d denotes dihydroxy and t trihydroxy. Thus d18:1 designates sphingosine (1,3-dihydroxy-2-aminooctadecene) and t18:0 phytosphingosine (1,3,4-trihydroxy-2-aminooctadecene).

Even though the description, examples and claims only mention Helicobacter pylori, other very similar Helicobacter species are also included in the scope of the present invention.

The invention is further illustrated in the examples below, which in no way are intended to limit the scope of the invention.

### BRIEF DESCRIPTION OF THE DRAWINGS

In the examples below, reference is made to the appended drawings on which:

FIG. 1 illustrates the binding of ³⁵S-labeled Helicobacter pylori to glycosphingolipids separated by thin-layer chromatography. FIG. 1 (A) illustrates glycosphingolipids detected with anisaldehyde reagent. FIG. 1 (B) and FIG. 1 (C) illustrate glycosphingolipids detected by autoradiography after binding of radiolabelled Helicobacter pylori strain 17875. Lane 1=non-acid glycosphingolipids of human blood group A erythrocytes, lane 2=non-acid glycosphingolipids of dog small intestine, lane 3 non-acid glycosphingolipids of guinea pig small intestine, lane 4=non-acid glycosphingolipids of mouse faeces, lane 5=non-acid glycosphingolipids of epithelial cells of black-and-white rat small intestine, lane 6=non-acid glycosphingolipids of human meconium, lane 7=acid glycosphingolipids of human blood group O erythrocytes, lane 8=acid glycosphingolipids of rabbit thymus, lane 9=gangliosides of calf brain, lane 10=acid glycosphingolipids from human hypernephroma. The designations to the left of (A) indicate the number of carbohydrate residues in the bands.

FIG. 2 illustrates a mass spectrum of the permethylated Helicobacter pylori-binding glycosphingolipid isolated from human meconium. Above the spectrum is a simplified formula representing the ceramide species with sphingosine and hydroxy 24:0 fatty acid.

FIG. 3 illustrates the anomeric region. of a proton NMR spectrum of the glycosphingolipid from human meconium. 4000 scans were collected at a probe temperature of 30° C. The large dispersion like signal at 5.04 ppm is an instrumental artifact, and there is also an unidentified impurity at 4.93 ppm.

FIG. 4 illustrates the binding of Helicobacter pylori to pure glycosphingolipids separated on thin-layer plates. Lane 1=lactotriaosylceramide, lane 2=lactotetraosylceramide, lane 3=H5 type 1 glycosphingolipid, lane 4=Le^{a}-5 glycosphingolipid, lane 5=Le^{b}-6 glycosphingolipid, lane 6=X-5 glycosphingolipid, lane 7=Y6 glycosphingolipid, lane 8=B6 type 1 glycosphingolipid. FIG. 4 A shows chemical detection by anisaldehyde, and FIG. 4 B is an autoradiogram obtained by binding of ³⁵S-labeled Helicobacter pylori.

FIG. 5 illustrates the effect of preincubation of Helicobacter pylori with the oligosaccharides lactose and lactotetraose. FIG. 5 A is a thin-layer chromatogram stained with anisaldehyde, FIG. 5 B shows binding of Helicobacter pylori incubated with lactose, and FIG. 5 C shows binding of Helicobacter pylori incubated with lactotetraose. Lane 1=gangliotetraosylceramide, lane 2=lactotetraosylceramide, lane 3=neolactotetraosyl-ceramide.

FIG. 6 illustrates a thin-layer chromatogram of separated glycosphingolipids detected with anisaldehyde (FIG. 6 A) and an autoradiogram obtained by binding of ³⁵S-labeled Helicobacter pylori strain 002 (FIG. 6 B) Lane 1=lactotetraosylceramide of human meconium, lane 2=non-acid glycosphingolipids of human meconium, lane 3=non-acid glycosphingolipids of human stomach of a blood group A(Rh+)p individual, lane 4=non-acid glycosphingolipids of human stomach of a blood group A(Rh+)P individual. The number of carbohydrate residues in the bands are indicated by the designations to the left.

FIG. 7 illustrates binding of Helicobacter pylori to non-acid glycosphingolipids from the epithelial cell of human stomach. Lane 1=reference non-acid glycosphingolipids of dog small intestine, lane 2=reference nonacid glycosphingolipids of mouse faeces, lane 3=reference non-acid glycosphingolipids of human meconium, lanes 4-8=non-acid glycosphingolipids (80 μg/lane) of epithelial cell of human stomach of five individuals (cases 1-5 of Table III). FIG. 7 A illustrates chemical detection with anisaldehyde, and FIG. 7 B is an autoradiogram obtained by binding of ³⁵S-labelled Helicobacter pylori. The number of carbohydrate residues in the bands are indicated by the designations to the left.

FIG. 8 is a thin-layer chromatogram showing the tetraglycosylceramide-containing fractions obtained from the epithelial cells of the stomach of case 4 and 5 of Table III (A), and the anomeric regions of 500 MHz proton NMR spectra of fraction 4-II (B) and 5-II (C). Lane 1=total non-acid glycosphingolipids of the stomach epithelium of case 4, lane 2=fraction 4-I from case 4, lane 3=fraction 4-II from case 4, lane 4=total non-acid glycosphingolipids of the stomach epithelium of case 5, lane 5=fraction 5-I from case 5, lane 6=fraction 5-II from case 5. The number of carbohydrate residues in the bands are indicated by the designations to the left.

FIG. 9 shows reconstructed ion chromatograms of permethylated oligosaccharides released by ceramide glycanase. Run A=reference mixture of globoside, lactotetraosylceramide and lactoneotetraosylceramide, run B=the tetraglycosylceramides from the stomach epithelium of case 4 of Table III, run C=the tetraglycosylceramides from the stomach epithelium of case 5 of Table III. The oligosaccharides of the reference mixture (Run A) have been marked.

FIG. 10 shows mass spectra obtained by high-temperature gas chromatography—EI mass spectrometry of permethylated oligosaccharides released by ceramide glycanase from reference glycosphingolipids (I and II), tetraglycosylceramide fraction from the stomach epithelium of case 4 of Table III (III), and tetraglycosylceramide fraction from the stomach epithelium of case 5 of Table III (IV).

FIG. 11 illustrates lactotetraosylceramide recognition both by the sialic acid-binding H. pylori strain CCUF 17874 (B) and the strain CCUG 17875 which is devoid of sialic acid binding capacity (C).

FIG. 12 shows the minimum energy conformers of the Helicobacter pylori-binding lactotetraosylceramide (FIG. 12 A), and the non-binding Le^{a}-5 glycosphingolipid (B), Le^{b}-6 glycosphingolipid (C) and defucosylated B6 type 1 glycosphingolipid (D).

FIG. 13 shows molecular models of minimum energy conformers of lactotetraosylceramide and gangliotetraosylceramide showing that the terminal disaccharide may be presented identically by varying only the Glcβ1Cer dihedral angles. Generation of all possible low-energy conformations having variant dihedral angles over the Glcβ1Cer linkage (Φ, Ψ and θ) for lactotetraosylceramide and gangliotetraosylceramide, followed by a pairwise comparison of the respective conformers, shows that two pairs are obtained in which the terminal disaccharide has the same orientation for these two glycosphingolipids. In the first pair the lactotetraosylceramide (A) dihedral angles over the Glcβ1Cer linkage are 51, −179 and 67, while for gangliotetraosylceramide (B) the same angles are 51, 180 and 177. The conformation in (A) is stabilised by an intramolecular hydrogen bond between the 2-OH of Glc and 3-O of the long-chain base, whereas the conformation in (B) is referred to as the extended one. In the second pair the Glcβ1Cer dihedral angles for lactotetraosylceramide (C) are 13, −90 and −59 and for gangliotetraosylceramide (D) 53, −173 and −64 . In the lactotetraosylceramide case the 2-OH of Glc forms a hydrogen bond with the 2-OH of the fatty acid and the NH of the long-chain base whereas gangliotetraosylceramide has the same Glcβ1Cer conformation as found in the crystal structure of Galβ1Cer. The methyl carbon of the acetamido groups of GlcNAc/GalNAc is shown in black.

### EXAMPLES

The abbreviations used in the examples are the following:

CFU=colony forming units;

Hex=hexose;

HexN=N-acetylhexosamine;

EI=electron ionization.

In the examples, the binding of Helicobacter pylori to glycosphingolipids is examined by binding of ³⁵S-labeled bacteria to glycosphingolipids on thin-layer chromatograms. Two separate binding specificities were frequently detected; on one hand a binding of Helicobacter pylori to lactosylceramide, gangliotriaosylceramide and gangliotetraosylceramide, and on the other, a selective binding to a non-acid tetraglycosylceramide from human meconium. The latter Helicobacter pylori-binding glycosphingolipid was isolated and, on the basis of mass spectrometry, proton NMR spectroscopy, and degradation studies, identified as Galβ3GlcNAcβ3Galβ4Glcβ1Cer (lactotetraosylceramide). Binding of Helicobacter pylori to the tetraglycosylceramide region of the non-acid glycosphingolipid fraction from gastric epithelial cells was obtained in one of seven human individuals, and the presence of lactotetraosylceramide in this fraction was confirmed by proton NMR spectroscopy and gas chromatography—EI mass spectrometry of permethylated tetrasaccharides obtained by ceramide glycanase treatment. The expression of the lactotetraosylceramide binding property was detected in 57 of 66 Helicobacter pylori isolates (86%).

Materials and Methods

Bacterial Strains, Culture Conditions and Labelling

The bacteria used, and their sources, are described in Table I at the end of the description part. In most of the experiments four strains, type strain 17875 (obtained from Culture Collection, University of Göteborg, (CCUG), Sweden, and the clinical isolates 002, 032 and 306, were used in parallel.

The strains were stored at −80° C. in tryptic soy broth containing 15% glycerol (by volume), and were initially grown on GAB-CAMP agar (19) in a humid (98%) microaerophilic atmosphere (5-7% 02, 8-10% C02, 85% N2) at 37° C. for 48-72 h. For labelling, colonies were inoculated on GAB-CAMP, or Brucella, agar plates and 50 μCi ^{[35]}S- methionine (Amersham, UK) diluted in 0.5 ml phosphate-buffered saline (PBS), pH 7.3, was sprinkled over the plates. After incubation for 12-36 h at 37° C. under microaerophilic conditions the cells were scraped off, washed three times with PBS, and resuspended in PBS to 1×10⁸ CFU/ml.

Alternatively, colonies were inoculated (1×10⁵ CFU/ml) in Ham's F 12 medium (Gibco BRL, UK), supplemented with 10% heat-inactivated foetal calf serum (Seralab, Göteborgs Termometerfabrik, Sweden) and 50 μCi ^{[35]}S-methionine. The culture bottles were incubated with shaking under microaerophilic conditions at 37° C. for 24 h. Aliquots from the culture bottles were tested for urease-, oxidase-, and catalase-activity and examined by phase-contrast microscopy to ensure a low content of coccoidal forms. Bacterial cells were harvested by centrifugation, and after two washes with PBS, the cells were resuspended to 1×10⁸ CFU/ml in PBS.

Both labelling procedures resulted in suspensions with specific activities of approximately 1 cpm per 100 Helicobacter pylori organisms.

Extraction of Bacterial Surface Proteins

Before the extraction procedure, Helicobacter pylori strains (denoted with * in Table I) were cultured on 5% horse blood agar under microaerophilic conditions at 37° C. for 2-3 days, harvested and washed once with PBS. Crude extracts were prepared by incubating bacterial cells with 1 M LiCl in PBS at 45° C. for 2 h (20). After centrifugation, the supernatants were dialysed overnight against PBS. The protein concentrations of the extracts were 300-1500 μg/ml, as determined by using an acidic solution of Coomassie Brilliant Blue G-250 dye reagent (Bio-Rad, Richmond, UK). From each extract aliquots of approximately 100 μg protein were taken out, and labelled with ^{[125]}I by the Iodogen method (21), to a specific activity of 2-5×10³ cpm/μg.

Thin-Layer Chromatography

Thin-layer chromatography was performed on glass- or aluminium-backed silica gel 60 HPTLC plates (Merck, Darmstadt, Germany), using chloroform/methanol/water (60:35:8, by volume) or chloroform/methanol/water containing 0.02% CaCl2 (60:40:9, by volume) as solvent systems. Chemical detection was accomplished by anisaldehyde (22) or the resorcinol reagent (23).

Chromatogram Binding Assay

The chromatogram binding assays were done as described (24). Mixtures of glycosphingolipids (20-80 μg/lane) or pure compounds (1-4 μg/lane) were separated on aluminum-backed silica gel 60 HPTLC plates. The dried chromatograms were soaked for 1 min in diethylether/n-hexane (1:5, by volume) containing 0.5% (w/v) polyisobutylmethacrylate (Aldrich Chem. Comp. Inc., Milwaukee, Wis.). After drying, the chromatograms were coated in order to block unspecific binding sites. Initially different coating conditions were tested, e.g. 1% polyvinylpyrrilidone (w/v) in PBS (Solution 1), 2% gelatine (w/v) in PBS (Solution 2), 2% bovine serum albumin (w/v) in PBS (Solution 3), 2% bovine serum albumin (w/v) and 0.1% (w/v) Tween 20 in PBS (Solution 4), or 2% bovine serum albumin (w/v) and 0.2% (w/v) deoxycholic acid in PBS (Solution 5). The most consistent results were obtained with Solution 4, which subsequently was used as the standard condition. Coating was done for 2 h at room temperature. Thereafter a suspension of 35S-labeled bacteria (diluted in PBS to 1×10 g CFU/ml and 1-5×10⁶ cpm/ml) or 125I-labeled bacterial surface proteins (diluted in Solution 4 to approximately 2×106 cpm/ml) were gently sprinkled over the chromatograms and incubated for 2 h at room temperature. After washing six times with PBS, and drying, the thin-layer plates were autoradiographed for 3-120 h at room temperature, or at −70° C., using XAR-5 x-ray films (Eastman Kodak, Rochester, N.Y.).

Glycosphingolipid Preparations

Reference Glycosphingolipids

Acid and non-acid glycosphingolipid fractions, from the sources given in Table II at the end of the description part, were obtained by standard procedures (25). The individual glycosphingolipids were isolated by acetylation of the total glycosphingolipid fractions and repeated chromatography on silicic acid columns. The identity of the purified glycosphingolipids was confirmed by mass spectrometry (26), proton NMR spectroscopy (27-30), and degradation studies (31, 32).

Galβ3GlcNH₂β3Galβ4Glcβ1Cer (No. 3 in Table II) was generated from Galβ3GlcNAcβ3Galβ4Glcβ1Cer (No. 2 in Table II) by treatment with anhydrous hydrazine, as described in (16).

Human Meconium

Meconia were pooled from 17 newborn full-term children, delivered at the Obstetric Clinic, Sahlgrenska University Hospital, Göteborg. Only the first portion passed within 24 h after delivery was collected and, after lyophilisation, kept at −70° C. Non-acid glycosphingolipids were isolated from the pooled material (dry weight 23.3 g) as described (25). Briefly, the lyophilised material was extracted in two steps in a Soxhlet apparatus with mixtures of chloroform and methanol (2:1 and 1:9, by volume, respectively) . The pooled extracts were subjected to mild alkaline methanolysis and dialysis, followed by separation on a silicic acid column (Mallinckrodt Chem. Work, St. Louis). Acid and non-acid glycolipid fractions were obtained by chromatography on a DEAE-cellulose column (DE-23, Whatman). In order to remove alkali-stable phospholipids from the non-acid glycolipids, this fraction was acetylated (24) and separated on a second silicic acid column, followed by deacetylation and dialysis. After final purification on DEAE-cellulose- and silicic acid columns 262 mg non-acid glycosphingolipids were obtained.

Isolation of the Helicobacter pylori-binding glycosphingolipid was performed by a two-step procedure. First, 240 mg of the non-acid glycosphingolipid fraction were separated by HPLC on a 2.2×30 cm column of silica (YMC SH-044-10, 10 μm particles; Skandinaviska Genetec, Kungsbacka, Sweden). The column was equilibrated in chloroform/methanol/water (65:25:4, by volume) (solvent A) and eluted (2 ml/min) with linear gradients of chloroform/methanol/water (40:40:12, by volume, solvent B) in solvent A. The column was first eluted with solvent A for 2 min, then the percentage of solvent B in solvent A was raised from 0% to 50% during 5 min, from 50% to 80% during 140 min, from 80% to 100% during 10 min, and kept at 100% during 23 min. Aliquots of each 2 ml fraction were analysed by thin-layer chromatography, and the fractions positive for anisaldehyde staining were further tested for binding of Helicobacter pylori, using the chromatogram binding assay. The Helicobacter pylori-binding fractions were collected in tubes 78-88, and after pooling of these fractions 14.2 mg were obtained.

This material was acetylated, and further separated by HPLC on an YMC SH-044-10 column. The column, equilibrated in chloroform, was eluted with a flow rate of 2 ml/min, with linear gradients of chloroform/methanol (95:5, by volume) (solvent C) in chloroform. The percentage of solvent C in chloroform was raised from 0% to 20% during 10 min, from 20% to 100% during 70 min, and kept at 100% during 10 min. After deacetylation, aliquots from each 1 ml fraction were analysed by anisaldehyde staining on thin-layer chromatograms, and the glycosphingolipid-containing fractions were examined for Helicobacter pylori-binding activity. Most of the Helicobacter pylori-binding glycosphingolipid was collected in tube 62, and this fraction (2.4 mg) was used for structural characterisation.

Epithelial Cells of Human Stomach

Stomach tissue (10×10 cm pieces) were obtained from the fundus region from patients undergoing elective surgery for morbid obesity. After washing with 0.9% NaCl (w/v), the mucosal cells were gently scraped off, and kept at −70° C. The material was lyophilised, and acid and non-acid glycosphingolipids were isolated as described (25) In two cases glycosphingolipids were also isolated from the nonmucosal residues. The blood group of the patients, and the amounts of glycosphingolipids isolated from each tissue specimen, are given in Table III at the end of the description part.

The non-acid glycosphingolipids from case 4 in Table III (2.9 mg) were separated by HPLC on a 1.0×25 cm silica gel column (Kromasil-Sil, 10 μm particles, Skandinaviska Genetec) using a gradient of chloroform/methanol/water (65:25:4 to 40:40:12, by volume) over 180 min, with a flow rate of 2 ml/min. Aliquots from each fraction were analysed by thin-layer chromatography using anisaldehyde as staining reagent. The tetraglycosylceramides were collected in tubes 12-17. Tubes 12-14 also contained a compound with mobility in the triglycosylceramide region on thinlayer chromatograms, and after pooling of these three fractions 0.2 mg was obtained (designated fraction 4-I). The fractions in tubes 15-17 were pooled separately giving 0.5 mg of tetraglycosylceramides (designated fraction 4-II).

Separation of 10.0 mg of the non-acid glycosphingolipid fraction from case 5 was done using the same system as above, with a gradient formed from chlorform/methanol/water (60:35:8 to 40:40:12, by volume). The fraction collected in tube 11 (designated fraction 5-I) contained triglycosylceramides and tetraglycosylceramides (0.1 mg), while only tetraglycosylceramides were obtained in tube 12 and 13. Pooling of the latter two fractions resulted in 0.3 mg (designated fraction 5-II).

EI Mass Spectrometry

Before mass spectrometry, the glycosphingolipids were permethylated, using solid NaOH in dimethyl sulfoxide and iodomethane, as described (33). The tetraglycosylceramide isolated from human meconium was analysed on a VG ZAB 2F/I-IF mass spectrometer (VG Analytical, Manchester, UK), using the in beam technique (34). Conditions for the analysis are given in the legend of the reproduced spectrum. The tetraglycosylceramides from the mucosal cells of human stomach were analysed by the same technique on a JEOL SX102A mass spectrometer (JEOL, Tokyo, Japan). Analytical conditions were: electron energy 70 eV, trap current 300 μA, and acceleration voltage 10 kV. The temperature was raised by 15° C./min, starting at 150° C.

Degradation Studies

The permethylated glycosphingolipid from human meconium was hydrolysed, reduced and acetylated (31, 32), and the partially methylated alditol- and hexosaminitols acetates obtained were analysed by gas chromatography—EI mass spectrometry on a Trio-2 quadrupole mass spectrometer (VG Masslab, Altrincham, UK). The Hewlett Packard 5890A gas chromatograph was equipped with an on-column injector and a 15 m×0.25 mm fused silica capillary column, DB-5 (J&W Scientific, Ranco Cordova, Calif.), with 0.25 μm film thickness. The samples were injected on-column at 70° C. (1 min) and the oven temperature was increased from 70° C. to 170° C. at 50° C./min, and from 170° C. to 260° C. at 8° C./min. Conditions for mass spectrometry were: electron energy 40 eV, trap current 200 μA. The components were identified by comparison of retention times and mass spectra of partially methylated alditol acetates obtained from reference glycosphingolipids.

Proton NMR Spectroscopy

Proton NMR spectra were acquired at 7.05 T (300 MHz) on a Varian VXR 300 (Varian, Palo Alto, Calif.) and at 11.75 T (500 MHz) on a JEOL Alpha-500 (JEOL, Tokyo, Japan). Data were processed off line using NMR1 (NMRi, Syracuse, N.Y.). The deuterium exchanged glycosphingolipid fractions were dissolved in dimethylsulfoxide-d₆/D₂O (98:2, by volume), and spectra were recorded at 30° C. with a 0.4 Hz digital resolution. Chemical shifts are given relative to tetramethylsilane.

Inhibition with Soluble Oligosaccharides

As a test for possible inhibition of binding by soluble sugars 35S-labeled Helicobacter pylori strains 002 and 032 were incubated for 1 h at room temperature with various concentrations (0.05 mg/ml, 0.1 mg/ml and 0.2 mg/ml) of lactotetraose (Accurate Chem. & Sci. Corp., Westbury, N.Y.) or lactose (J. T. Baker Chem. Co., Phillipsburg, N.J.) in PBS. Thereafter the chromatogram binding assay was performed as described above, using chromatograms with separated gangliotetraosylceramide, lactotetraosylceramide and reference glycosphingolipids.

Molecular Modelling

Minimum energy conformations of the various glycosphingolipids listed in Table II were calculated within the Biograf molecular modelling program (Molecular Simulations Inc., Waltham, Mass.) using the Deriding-II force field (35) on a Silicon Graphics4D/3STG workstation. Charges were generated using the charge equilibration method (36) and a distance dependent dielectric constant of ε=3.5 r was used for the Coulomb interactions. In addition a special hydrogen bonding term was used in which Dhb was set to −4 kcal/mol (35).

Ceramide Glycanase Treatment of Tetraglycosylceramides from Human Stomach Epithelium

The procedure of Hansson et al. (37) was used for the enzymatic hydrolysis. Briefly, 100 μg of fraction 4-II from case 4, fraction 5-II from case 5, reference globoside from human erythrocytes (38), reference lactotetraosylceramide from human meconium, and reference lactoneotetraosylceramide (obtained by sialidase treatment of sialyl-lactoneotetraosylceramide from human erythrocytes; Ref. 39) were dissolved in 100 μl 0.05 M sodium acetate buffer, pH 5.0, containing 120 μg sodium cholate, and sonificated briefly. Thereafter, 1 mU of ceramide glycanase from the leech, Macrobdella decora (Boehringer Mannheim, Mannheim, Germany) was added and the mixtures were incubated at 37° C. for 24 h. The reaction was stopped by addition of chloroform/methanol/water to the final proportions 8:4:3 (by volume). The oligosaccharide-containing upper phase thus obtained was separated from ceramides and detergent on a Sep-Pak C₁₈ cartridge (Waters, Milford, Mass.). The eluant containing the oligosaccharides was dried under nitrogen and under vacuum, and thereafter permethylated as described (33).

High-Temperature Gas Chromatography and Gas Chromatography—EI Mass Spectrometry of the Permethylated Oligosaccharides

The analytical conditions were essentially the same as described in (40). Capillary gas chromatography was performed on a Hewlett-Packard 5890A gas chromatograph using a fused silica column (10 m×0.25 mm i.d.) coated with 0.03 μm of crosslinked PS 264 (Fluka, Buchs, Switzerland), and with hydrogen as carrier gas. The permethylated oligosaccharides were dissolved in ethylacetate, and 1 μl of sample was injected on-column at 70° C. (1 min). A two-step temperature program was used; 70° C. to 200° C. at 50° C./min, followed by 10° C./min up to 350° C.

Gas chromatography—EI mass spectrometry was performed on a Hewlett-Packard 5890-II gas chromatograph coupled to a JEOL SX-102A mass spectrometer. The chromatographic conditions, as well as the capillary column, were the same as for the analyses by gas chromatography, and the conditions for mass spectrometry were: interface temperature 350° C., ion source temperature 330° C., electron energy 70 eV, trap current 300 μA, acceleration voltage 10 kV, mass range scanned 100-1600, total cycle time 1.4 sec, resolution 1000, and pressure in the ion source region 10⁻⁵ Pa.

Results

Binding to Mixtures of Reference Glycosphingolipids

A number of well characterised glycosphingolipid mixtures, representing a large variety of carbohydrate sequences, were separated by thin-layer chromatography.

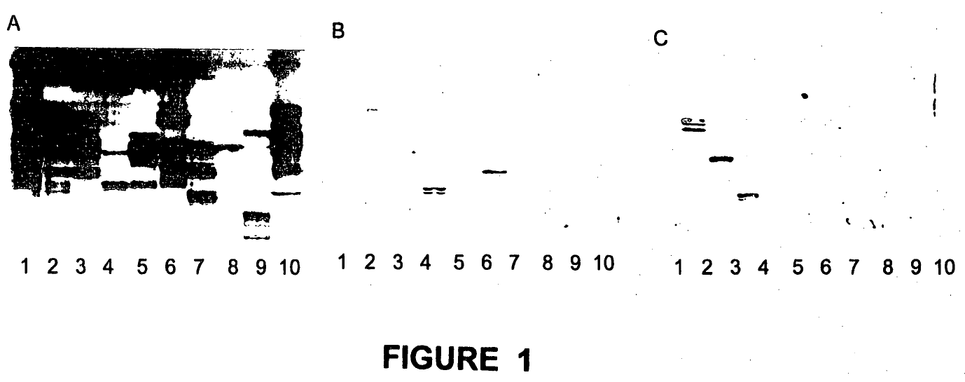

The results are shown in FIG. 1, which illustrates the binding of ³⁵S-labeled Helicobacter pylori or ¹²⁵I- labelled bacterial surface proteins to glycosphingolipids separated by thin-layer chromatography. FIG. 1 A illustrates glycosphingolipids detected with anisaldehyde reagent. By autoradiography after binding of radiolabelled Helicobacter pylori strain 17875 only a few selective bands were visualised, as shown in FIG. 1 B and FIG. 1 C. The same binding pattern was obtained with radiolabelled bacterial surface proteins (not shown). The glycosphingolipids were separated on aluminium-backed silica gel 60 HPTLC plates, using chloroform/methanol/water (60:35:8, by volume) as solvent system, and the binding assay was performed as described in “Materials and Methods”. The autoradiogram in FIG. 1 B was obtained after coating of the thin-layer chromatogram with 2% BSA and 0.1% Tween 20 in PBS, whereas the autoradiogram in FIG. 1 C was obtained when the coating buffer contained only 2% BSA in PBS. The lanes contained the following glycosphingolipids: non-acid glycosphingolipids of human blood group A erythrocytes, 40 μg (lane 1); non-acid glycosphingolipids of dog small intestine, 40 μg (lane 2); non-acid glycosphingolipids of guinea pig small intestine, 20 μg (lane 3); non-acid glycosphingolipids of mouse faeces, 20 μg (lane 4); non-acid glycosphingolipids of epithelial cells of black-and-white rat small intestine, 40 μg (lane 5); non-acid glycosphingolipids of human meconium, 40 μg (lane 6); acid glycosphingolipids of human blood group O erythrocytes, 40 μg (lane 7); acid glycosphingolipids of rabbit thymus, 20 μg (lane 8); gangliosides of calf brain, 40 μg (lane 9); acid glycosphingolipids from human hypernephroma, 40 μg (lane 10). Autoradiography was for 12 h.

The binding in lane 2 (lactosylceramide), lane 3 (gangliotriaosylceramide) and lane 4 (gangliotetraosylceramide), was judged to correspond to the “lactosylceramide binding specificity” and the “ganglio binding specificity” of Helicobacter pylori previously described in detail (16).

In addition, a selective binding of Helicobacter pylori to a component with mobility in the tetraglycosylceramide region in the non-acid glycosphingolipid fraction from human meconium was detected (FIG. 1, B, lane 6). The latter binding activity was only detected when the coating buffer contained detergent (Tween 20 or deoxycholic acid), as shown in FIG. 1. Solution 4 (2% bovine serum albumin and 0.1% Tween 20 in PBS) was subsequently utilised as standard coating procedure. The binding-active tetraglycosylceramide from human meconium was isolated by HPLC, and characterised by mass spectrometry, proton NMR spectroscopy, and gas chromatography—EI mass spectrometry after degradation, as follows.

Chemical Structure of the Helicobacter Pylori-Binding Glycosphingolipid from Human Meconium

The binding-active tetraglycosylceramide was isolated from 240 mg of total non-acid glycosphingolipids. By HPLC of the native glycosphingolipid mixture 14.2 mg of tetraglycosylceramides were obtained. This tetraglycosylceramide fraction was a complex mixture, and in addition to the Helicobacter pylori-binding compound it contained at least three other glycosphingolipids. The tetraglycosylceramide fraction was acetylated and further separated by HPLC, giving 2.4 mg of the pure binding-active glycosphingolipid. Each step during the preparative procedure was monitored by binding of radiolabelled Helicobacter pylori on thin-layer chromatograms.

EI Mass Spectrometry

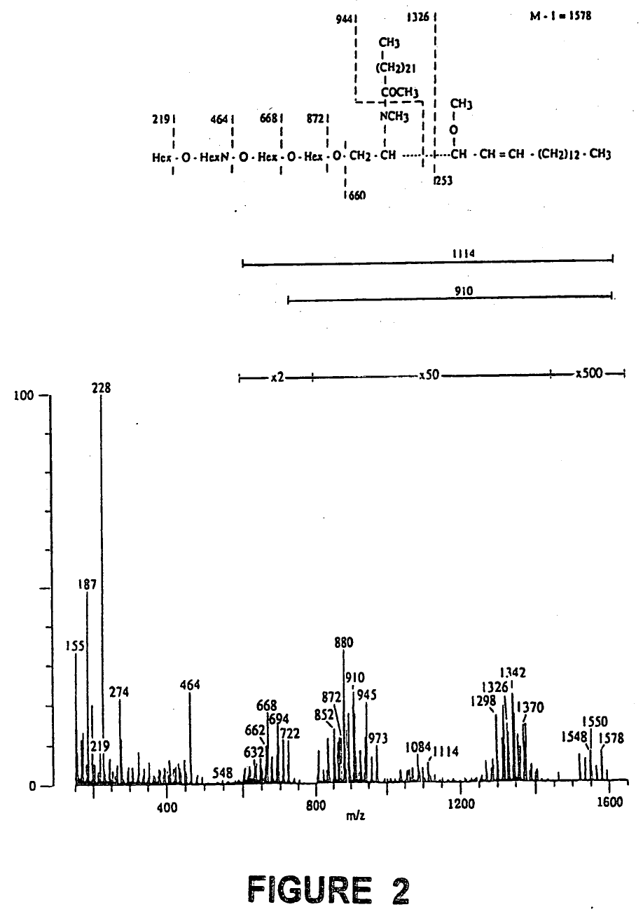

The mass spectrum of the permethylated binding-active glycosphingolipid from human meconium was also studied, together with a simplified formula for interpretation, representing the species with d18:1-h24:0 ceramide. The results are shown in FIG. 2. Above the spectrum is a simplified formula for interpretation, representing the ceramide species with sphingosine and hydroxy 24:0 fatty acid. Analytical conditions were: sample amount 16 μg, electron energy 45 eV, trap current 500 μA and acceleration voltage 8 kV. Starting at 250° C., the temperature was elevated by 6° C./min. The reproduced spectrum was recorded at 300° C.

The spectrum of the permethylated glycosphingolipid was dominated by oxonium ions, which give the carbohydrate sequence, and fragment ions due to inductive cleavage of the ceramide. The abundance of other fragment ions was very low but immonium ions, and in the case of phytosphingosine as long-chain base ions due to α-cleavage of the base, were present.

The immonium ions, formed by loss of part of the long-chain base, were found at m/z 1298 and 1326. These ions give information about the number and type of sugars and the fatty acid composition, and in the present case demonstrated the presence of one N-acetylhexosamine, three hexoses, combined with h22:0 and h24:0 fatty acids.

The carbohydrate sequence ions seen at m/z 219 and 187 (219 minus 32), 464, 668 and 872 demonstrated that the glycosphingolipid was a tetraglycosylceramide with the carbohydrate sequence Hex-HexN-Hex-Hex. This was supported by the fragment ion at m/z 945 (944+1), which consisted of the whole carbohydrate chain and part of the fatty acid. A type 1 chain (Hexβ-3HexN) was indicated by the absence of a fragment ion at m/z 182, which is a dominating ion in the case of 4-substituted HexN (41, 42). The intense fragment ion at m/z 228 was a secondary fragment from the internal HexN, since no terminal HexN at m/z 260 was found.

The molecular region was weak. However, [M−H]⁺ ions corresponding to the species with d 18:1-24:0, d 18:1-h22:0 and d 18:1-h24:0 ceramides, were found at m/z 1548, 1550 and 1578, respectively. Loss of terminal parts of the carbohydrate chain from the molecular ions were also seen (explained below the formula for the species with d18:1-h24:0 ceramide). The ions at m/z 1342 and 1370 were probably due to cleavage between the two hydroxy groups of the t18:0 long-chain base, of the t18:0-h22:0 and t18:0-h24:0 ceramide species, respectively.

Further information about the ceramide composition was given by the series of fragment ions at m/z 548-722, demonstrating a mixture of species ranging from d 18:1-16:0 to t 18:0-h24:0. The dominating ceramide species were d 18:1-24:0, d 18:1-h24:0, t 18:0-h22:0 and t 18:0h24:0, as judged from the relative intensities of the ceramide ions, the immonium ions, and molecular ions.

Thus, mass spectrometry of the permethylated glycosphingolipid demonstrated a carbohydrate chain with the sequence Hex-HexN-Hex-Hex, and d 18:1 and t 18:0 long-chain bases combined both hydroxy and non-hydroxy fatty acids, with mainly 22 and 24 carbon atoms.

Degradation Studies

The binding positions between the carbohydrate residues were obtained by degradation of the permethylated tetraglycosylceramide, i.e. the sample was subjected to acid hydrolysis, followed by reduction and acetylation. The resulting partially methylated alditol acetates were analysed by gas chromatography—EI mass spectrometry. The reconstructed ion chromatogram thus obtained had four carbohydrate peaks (not shown). The acetate of 2,3,4,6tetramethyl-galactitol identified a terminal galactose, while the presence of the acetate of 4,6dimethyl-2-N methyl-acetamido-glucitol (3-substituted N-acetylglucosamine), indicated a type 1 chain. The two remaining peaks, acetates of 2,4,6-trimethylgalactitol and 2,3,6-trimethylglucitol, were identified as 3-substituted galactose and 4-substituted glucose, respectively.

In combination with the data from mass spectrometry, a carbohydrate chain with the sequence Gal1-3GlcNAcβ-3 Gal1-4Glc1 could thus be deduced.

Proton NMR Spectroscopy

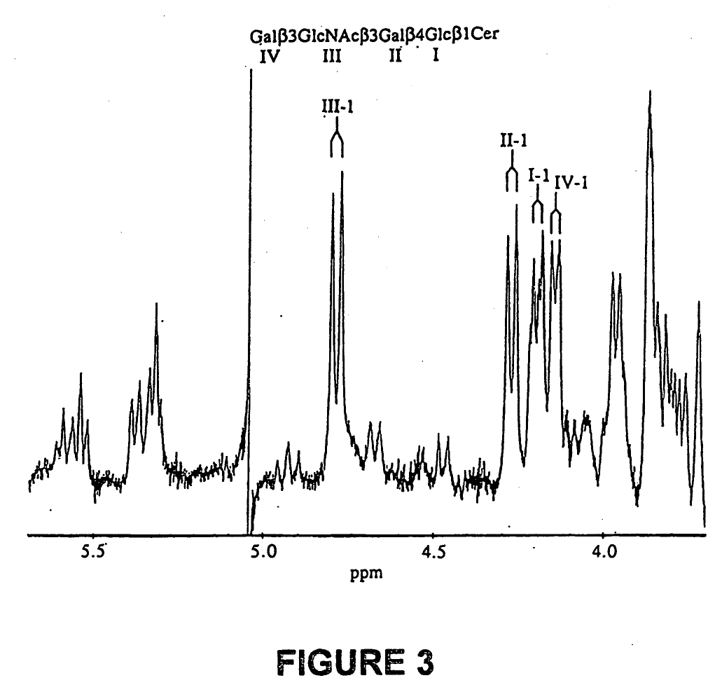

Thereafter a 300 MHz protone NMR spectroscopic study of the glycosphingolipid from human meconium was performed, and the results are shown in FIG. 3. 4000 scans were collected at a probe temperature of 30° C. The large dispersion like signal at 5.04 ppm is an instrumental artifact. There is also an unidentified impurity at 4.93 ppm.

The anomeric region of the proton NMR spectrum contained five large (3-doublets (J1,2≈8 Hz). The glucose anomeric proton signal (4.20 ppm, J_{1,2}=7.2 Hz) was split into two signals, as is often the case, due to ceramide head group differences. At 4.28 ppm (J_{1,2=7.2} Hz) the Gal(i4 anomeric proton appeared, which is indicative of a substitution at the 3-position. The internal GlcNAcβ anomer was seen at 4.79 ppm (J_{1,2}=8.0 Hz) with its N-acetamido methyl protons resonating at 1.82 ppm. Finally, the terminal Gal(3 signal was found at 4.15 ppm (J_{1,2}=6.6 Hz) indicating a 1-to-3 linkage. All anomeric chemical shifts were thus in agreement with published results for lactotetraosylceramide (45). In addition to the main compound, a small impurity was noted by the β-doublets at 4.67 and 4.47 ppm, seemingly corresponding to a lactogangliotetraosylceramide hybrid structure described in undifferentiated murine leukaemia cells (44).

From all the data combined, the structure of the Helicobacter pylori-binding glycosphingolipid from human meconium was established as Galβ3GlcNAcβ3Galβ4Glcβ1Cer, i.e. lactotetraosylceramide, which has previously been identified from the same source (45). The predominant ceramide species in the present case (mainly d18:1-24:0, d18:1-h24:0, t18:0-h22:0 and t18:0-h24:0) differed from the previous description, where only hydroxy fatty acids were found.

Comparison with Isoreceptors on Thin-Layer Chromatograms

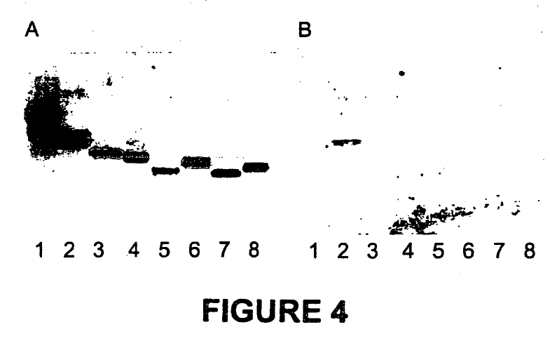

A number of pure glycosphingolipids, structurally related to lactotetraosylceramide, were examined for Helicobacter pylori-binding activity using the chromatogram binding assay. The results are summarised in Table II, and shown in FIG. 4. The lanes in FIG. 4 are the following: GlcNAcβ3Galβ4Glcβ1Cer (lactotriaosylceramide), 4 μg (lane 1); Galβ3GlcNAcβ3Galβ4Glcβ1Cer (lactotetraosylceramide), 4 μg (lane 2); Fucα2Galβ3GlcNAcβ3Galβ4Glcβ1Cer (H5 type 1 glycosphingolipid), 4 μg (lane 3);

Galβ3(Fucα4)GlcNAcβ3Galβ4Glcβ1Cer (Le^{a}-5 glycosphingolipid), 4 μg (lane 4);

Fucα2Galβ3(Fucα4)GlcNAcβ3Galβ4Glcβ1Cer (Le^{b}-6 glycosphingolipid), 4 μg (lane 5);

Galβ4(Fucα3)GlcNAcβ3Galβ4Glcβ1Cer (X-5 glycosphingolipid), 4 μg (lane 6);

Fucα2Galβ4(Fucα3)GlcNAcβ3Galβ4Glcβ1Cer (Y-6 glycosphingolipid), 4 μg (lane 7;

Galα3(Fucα2)Galβ3GlcNAcβ3Galβ4Glcβ1Cer (B6 type 1 glycosphingolipid), 4 μg (lane 8).

FIG. 4 A shows chemical detection by anisaldehyde, whereas FIG. 4 B shows an autoradiogram obtained by binding of ³⁵S-labeled Helicobacter pylori strain 032. The glycosphingolipids were separated on aluminium-backed silica gel 60 HPTLC plates, using chloroform/methanol/water (60:35:8, by volume) as solvent system, and the binding assay was performed as described under “Materials and Methods”, using 2% BSA and 0.1% Tween 20 in PBS, as coating buffer. Autoradiography was for 12 h.

The only binding-active glycosphingolipid was lactotetraosylceramide (No. 2), while all the substitutions tested abolished the binding. Thus, the addition of an α-fucose in 2-position (No. 4 of Table II), an α-N-glycolylneuraminic acid (No. 11) or an α-galactose (No. 8) in 3position of the terminal galactose, or an α-fucose in 4-position of the N-acetylglucosamine (No. 5), was not tolerated. No binding to the GlcNAcβ3Galμ4Glcβ1Cer glycosphingolipid (No. 1) was obtained, demonstrating the importance of the Galβ3GlcNAcγ-part. The acetamido group at 2-position of the penultimate N-acetylglucosamine contributed substantially to the interaction, since removal of this moiety (No. 3) completely abolished the binding.

Inhibition of Binding on Thin-Layer Chromatograms

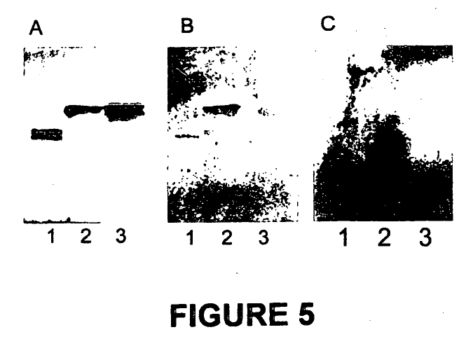

The ability of soluble oligosaccharides to interfere with the binding of Helicobacter pylori to glycosphingolipids on thin-layer plates was examined by incubating radiolabelled Helicobacter pylori strain 17875 with free lactotetraose (0.1 mg/ml) or lactose (0.2 mg/ml) in PBS for 1 h at room temperature before the chromatogram binding assay of the suspensions. The results are shown in FIG. 5. FIG. 5 A shows a thin-layer chromatogram stained with anisaldehyde, FIG. 5 B the binding of Helicobacter pylori incubated with lactose, and FIG. 5 C the binding of Helicobacter pylori incubated with lactotetraose. The lanes were: Galβ3GalNAcβ4Galβ4Glcβ1Cer (gangliotetraosylceramide), 4 μg (lane 1); Galβ3GlcNAcβ3Galβ4Glcβ1Cer (lactotetraosylceramide), 4 μg (lane 2); Galβ4GlcNAcβ3Galβ4Glcβ1Cer (neolactotetraosylceramide), 4 μg (lane 3). The glycosphingolipids were separated on aluminium-backed silica gel 60 HPTLC plates, using chloroform/methanol/water (60:35:8, by volume) as solvent system, and the binding assay was performed as described under “Materials and Methods”, using 2% BSA and 0.1% Tween 20 in PBS as coating buffer. Autoradiography was for 12 h.

Thus, incubation with lactotetraose (0.1 mg/ml) inhibited the binding of Helicobacter pylori to lactotetraosylceramide, while incubation with lactose had no inhibitory effect.

Binding of Helicobacter pylori to Glycosphingolipids of Human Stomach

Non-Acid Glycosphingolipids of Whole Human Stomach Wall

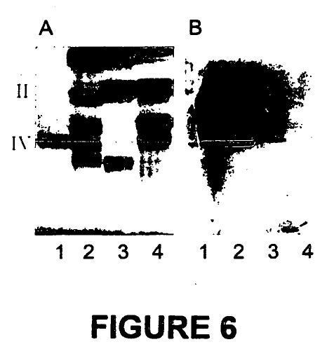

In order to examine the expression of binding-active glycosphingolipids in the target tissue of the bacteria, the binding of Helicobacter pylori to glycosphingolipids isolated from the whole human stomach wall was investigated, and the results are illustrated in FIG. 6, which shows a thin-layer chromatogram of separated glycosphingolipids detected with anisaldehyde (FIG. 6 A) and an autoradiogram obtained by binding of ³⁵S-labeled Helicobacter pylori strain 002 (FIG. 6 B) . The lanes were: lactotetraosylceramide of human meconium, 4 μg (lane 1); non-acid glycosphingolipids of human meconium, 40 μg (lane 2); non-acid glycosphingolipids of human stomach of a blood group A(Rh+)p individual, 40 μg (lane 3); non-acid glycosphingolipids of human stomach of a blood group A(Rh+)P individual, 40 μg (lane 4). The glycosphingolipids were separated on aluminium-backed silica gel 60 HPTLC plates, using chloroform/methanol/water (60:35:8, by volume) as solvent system, and the binding assay was done as described in the “Materials and Methods” section. The coating buffer contained 2% BSA and 0.1% Tween 20 in PBS. Autoradiography was for 5 h. The number of carbohydrate residues in the bands are indicated by the designations to the left.

The tetraglycosylceramide region of these non-acid fractions was dominated by globoside (exemplified in lane 4 of FIG. 6, A), which, at least for human small intestine (46) and colon (47), is derived from the nonepithelial stroma. No binding to these fractions was obtained (exemplified in FIG. 6, B, lane 4). However, when using the non-acid glycosphingolipid fraction isolated from the stomach of a blood group A(Rh+)p individual (48), which lacked the galactosyltransferase responsible for the conversion of lactosylceramide to globotriaosylceramide (49), and consequently was devoid of globoside (FIG. 6, A, lane 3), a binding of Helicobacter pylori in the tetraglycosylceramide region was detected (FIG. 6 B, lane 3). The tissue in this case was obtained after surgery for peptic ulcer disease. Due to limited amounts available, no chemical characterisation of this binding-active tetraglycosylceramide was possible.

Glycosphingolipids of Epithelial Cells of Human Stomach

Next the inventors examined the binding of Helicobacter pylori to glycosphingolipids isolated from the epithelial cells of human stomach. Since non-neoplastic pyloric tissue rarely is excised during normal surgical procedures, glycosphingolipids were isolated from specimens from the fundus region obtained from patients undergoing surgery for obesity, although this region of the stomach differ histologically from the pyloric region where Helicobacter pylori are most commonly found (50, 51).

In total, glycosphingolipids were isolated from mucosal scrapings from seven individuals, and in two cases also from the non-mucosal residues. Due to limited amounts of material, the binding to these fractions was only tested for the Helicobacter pylori strains 002 and 032.

The major compounds in acid glycosphingolipid fractions migrated on thin-layer chromatograms as sulfatide and GM3. No binding of Helicobacter pylori to these major acid glycosphingolipids was obtained (not shown). No binding of the bacteria to the glycosphingolipids from the nonepithelial stroma observed.

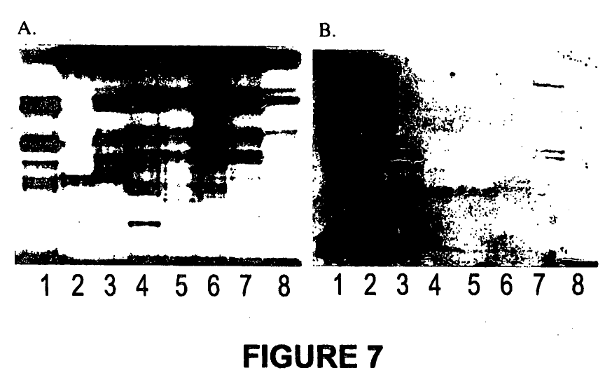

The binding of Helicobacter pylori to non-acid glycosphingolipid fractions isolated from the epithelial cells of human stomach from five of the seven cases was then studied, and the results are shown in FIG. 7 A, which illustrates chemical detection with anisaldehyde. In one of the seven individuals, a binding of Helicobacter pylori in the tetraglycosylceramide region was detected, as shown in FIG. 7 B. Lanes 1-3 in the figure are reference non-acid glycosphingolipids of dog small intestine, 40 μg (lane 1); mouse faeces, 20 μg (lane 2); human meconium, 40 μg (lane 3), while lanes 4-8 were non-acid glycosphingolipids (80 μg/lane) of epithelial cell of human stomach of five individuals (cases 1-5 of Table III). (B) Autoradiogram obtained by binding of ³⁵S-labelled Helicobacter pylori strain 032. The glycosphingolipids were separated on aluminium-backed silica gel 60 HPTLC plates, using chloroform/methanol/water (60:35:8, by volume) as solvent system, and the binding assay was performed as described under “Materials and Methods”, using 2% BSA and 0.1% Tween 20 in PBS as coating buffer. Autoradiography was for 12 h. The number of carbohydrate residues in the bands are indicated by the designations to the left.

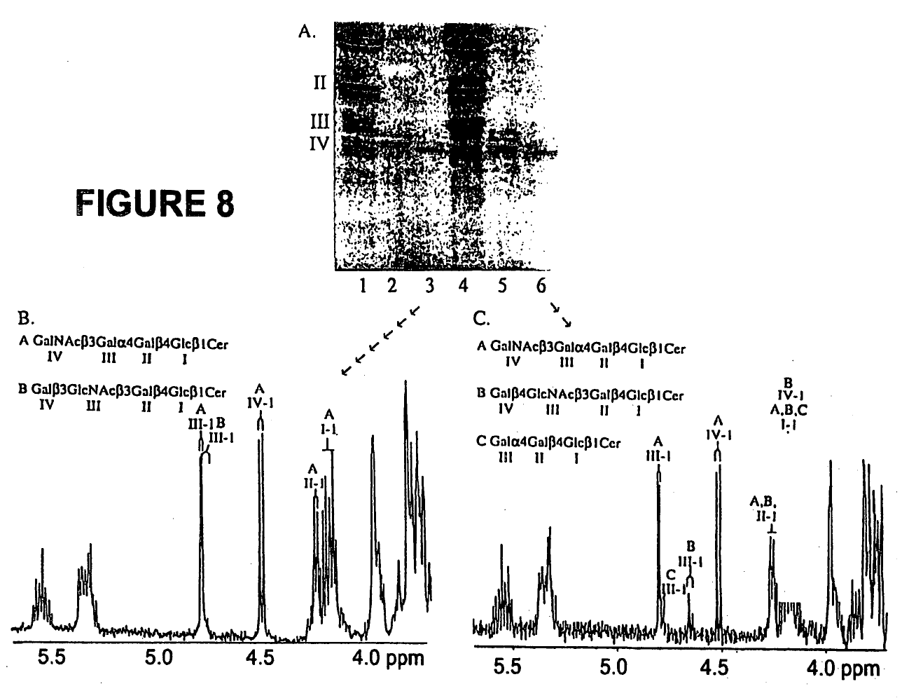

In addition, a binding-active compound with mobility in the diglycosylceramide region was found in one case, as described in a previous report (16). The fraction containing the binding-active tetraglycosylceramide (case 4), and one non-binding fraction (case 5), were separated by HPLC, and the isolated tetraglycosylceramides from each case were characterised by ¹H-NMR spectroscopy, EI mass spectrometry, and gas chromatography—EI mass spectrometry of permethylated tetrasaccharides obtained by hydrolysis with ceramide glycanase. The results are shown in FIG. 8, which is a thin-layer chromatogram showing the tetraglycosylceramide-containing fractions obtained from the epithelial cells of the stomach of case 4 and 5 of Table III (A), and the anomeric regions of 500 MHz proton NMR spectra of fraction 4-II (B) and 5-II (C). The lanes on the thin-layer chromatogram were: total non-acid glycosphingolipids of the stomach epithelium of case 4, 80 μg (lane 1); fraction 4-I from case 4, 4 μg (lane 2); fraction 4-II from case 4, 4 μg (lane 3); total non-acid glycosphingolipids of the stomach epithelium of case 5, 80 μg (lane 4); fraction 5-I from case 5, 4 μg (lane 5); fraction 5-II from case 5, 4 μg (lane μ The glycosphingolipids were separated on glass-backed silica gel 60 HPTLC plates, using chloroform/methanol/water (60:35:8, by volume) as solvent system, and stained with anisaldehyde. The number of carbohydrate residues in the bands are indicated by the designations to the left. For proton NMR spectroscopy, 4000 scans were collected from 0.5 mg (4-II) and 0.3 mg (5-II) of sample, respectively, at a probe temperature of 30° C.

Proton NMR Spectroscopy of the Tetraglycosylceramide Fractions from Epithelial Cells of Human Stomach

The proton NMR spectrum of fraction 4-II isolated from case 4 (FIG. 8 B) was dominated by globoside with its anomeric signals appearing at 4.81 ppm (Galα), 4.52 ppm (GalNAcβ), 4.26 ppm (Galβ) and 4.20/4.17 ppm (Glcβ). However, a small peak on the base of the Galα H1 signal revealed that also another glycosphingolipid was present in this fraction. This signal was consistent GlcNAcβ H-1 of lactotetraosylceramide, the potential other signals being buried under the globoside resonances. However, the Galα H1 of globotriaosylceramide would also have a very similar chemical shift. The exact shifts vary with temperature and other factors. To resolve this the inventors compared reference spectra of lactotetraosyl-, globotetraosyl-, and globotriaosylceramide run under similar conditions at 400 MHz. A reference mixture of lactotetraosylceramide and globotetraosylceramide was also prepared and run at 500 MHz. These comparisons clearly showed that the signal at 4.79 ppm belonged to a β-anomeric proton from the N-acetylglucosamine of lactotetraosylceramide. This was further corroborated when analysing the more early-eluting tetraglycosylceramide-containing fraction (4-I) from case 4. Here two non-overlapping α-anomeric signals from galactose, one corresponding to the internal Galα H1 of globotetraosylceramide (4.81 ppm), and the other corresponding to terminal Galα H1 of globotriaosylceramide (4.78 ppm), were found (not shown).

The presence of lactotetraosylceramide should also give rise to a different methyl signal from the N-acetamido glucose (52) compared to the N-acetamido galactose of globotria- and globotetraosylceramide. The GalNAc methyl signal was seen at 1.85 ppm and the methyl signal of the GlcNAc in lactotetraosylceramide at 1.82 ppm, which is identical to our reference spectra and in close agreement with the values reported in (53). From the intensities of the methyl signals it was estimated that fraction 4-II contained approximately 5% lactotetraosylceramide.

The early-eluting tetraglycosylceramide-containing fraction (5-I) from case 5 contained both globotria- and globotetraosylceramide, as evidenced by α-anomeric signals at 4.81 and 4.78 ppm, respectively (not shown). The more late-eluting tetraglycosylceramide-containing fraction (5-II), shown in FIG. 8, C, also contained a β-doublet at 4.65 ppm corresponding to GlcNAcβ of lactoneotetraosylceramide (53). The N-acetamido glucose of this glycosphingolipid had a methyl signal at 1.82 ppm, in agreement with earlier data on lactoneotetraosylceramide (52).

EI Mass Spectrometry of the Tetraglycosylceramide Fractions from Epithelial Cells of Human Stomach

The mass spectra (not shown) obtained by EI mass spectrometry of the permethylated derivatives of fraction 4-II and 5-II, from case 4 and 5 respectively, were very similar. In both spectra the ions at m/z 260 and 228 (260 minus 32) were prominent, demonstrating a terminal HexN, while no ion indicating a terminal Hex at m/z 219 was found. Terminal HexN-Hex was shown by an ion at m/z 464. A fragment ion at m/z 945 (944+1), containing the whole carbohydrate chain and part of the fatty acid, demonstrated a HexN-HexHex-Hex carbohydrate sequence.

From the relative intensities of the fragment ions from the ceramide part, immonium ions, and molecular ions, it was demonstrated that the predominant ceramide species of fraction 4-II was d18:1-16:0, d18:1-h24:0 and d18:1-h24:1, while fraction 5-II had mainly d18:1-16:0, d18:1-22:0, d18:1-24:0, d18:1-24:1, and d18:1-h24:0 ceramides.

Thus, by mass spectrometry only the major compound of the two samples, i.e. globoside was identified, while the minor compounds of the fractions indicated by the proton NMR experiments could not be discerned. However, the increased resolution obtained by combining chromatographic methods and mass spectrometry permitted the identification of these minor compounds, as described in the following part.

High Temperature Gas Chromatography—EI Mass Spectrometry of Permethylated Tetrasaccharides from Epithelial Cells of Human Stomach

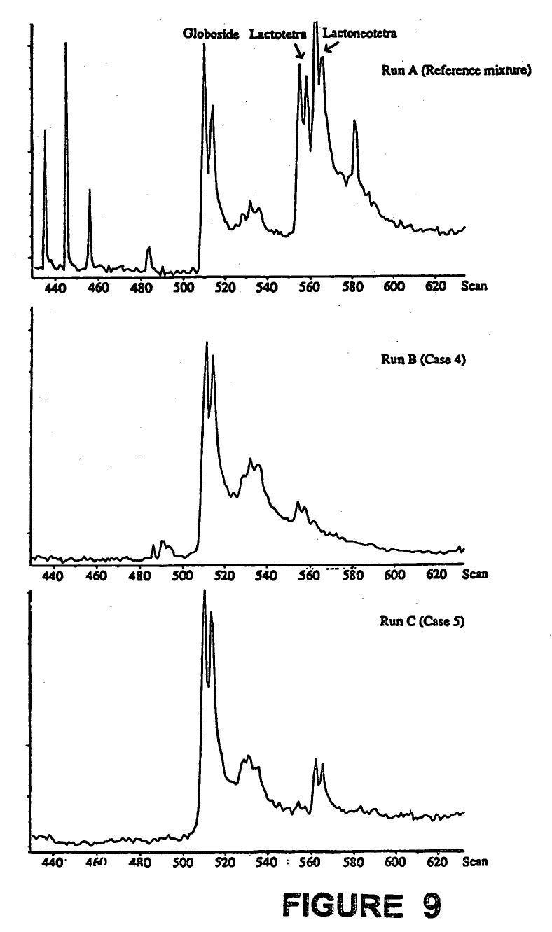

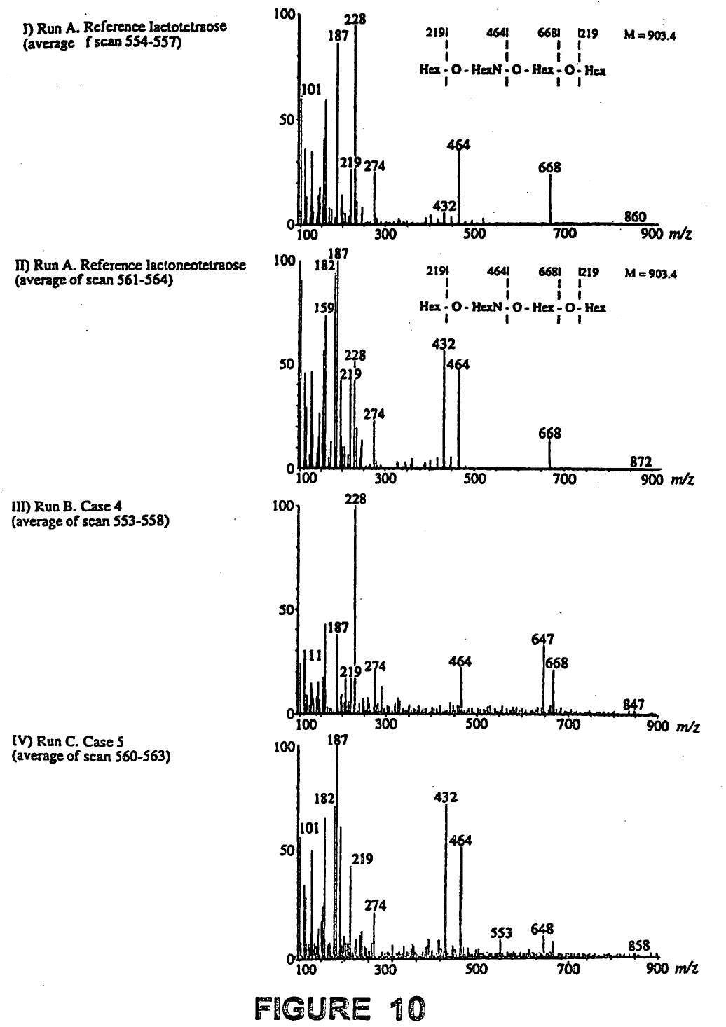

Fraction 4-II from case 4 and fraction 5-II from case 5 were hydrolysed with ceramide glycanase, and the released tetrasaccharides were permethylated and analysed by gas chromatography and gas chromatography—EI mass spectrometry. The results are summarised in FIGS. 9 and 10. Each chromatographic peak was resolved in α- and β-conformer.

FIG. 9 shows reconstructed ion chromatograms of permethylated oligosaccharides released by ceramide glycanase. Run A was a reference mixture of globoside, lactotetraosylceramide and lactoneotetraosylceramide, while run B was the tetraglycosylceramides from the stomach epithelium of case 4 of Table III, and run C was the tetraglycosylceramides from the stomach epithelium of case 5 of Table III. The analytical conditions are described in the “Materials and Methods” section. The oligosaccharides of the reference mixture (Run A) have been marked.

FIG. 10 shows mass spectra obtained by high-temperature gas chromatography—EI mass spectrometry of permethylated oligosaccharides released by ceramide glycanase from reference glycosphingolipids (I and II), tetraglycosylceramide fraction from the stomach epithelium of case 4 of Table III (III), and tetraglycosylceramide fraction from the stomach epithelium of case 5 of Table III (IV). For analytical conditions, see “Materials and Methods”. The designations Run A-C refer to the partial total ion chromatograms shown in FIG. 10. Interpretation formulae are shown together with the reference spectra.

The tetrasaccharides of the stomach epithelium of the Helicobacter pylori-binding case 4 were resolved into two peaks, as shown in FIG. 9, Run B. The dominating peak eluted at the same retention time as the saccharide from reference globoside, while the minor peak eluted at the retention time of the saccharide from reference lactotetraosylceramide.

The tetrasaccharides of the stomach epithelium of the non-binding case 5 (FIG. 9, Run C) were also resolved into two peaks, with the major peak at the same retention time as the saccharide from reference globoside. The smaller peak in this case eluted at the retention time of the saccharide of reference lactoneotetraosylceramide.

To further substantiate the differences in the tetraglycosylceramide fractions from the Helicobacter pylori-binding case 4 and the non-binding case 5, mass spectra of the permethylated oligosaccharides were obtained (FIG. 10). The spectra of the dominant peaks of both cases were in agreement with that of standard globoside (not shown). However, the spectra of the minor tetrasaccharides of the Helicobacter pylori-binding case 4 (FIG. 10, III), and the non-binding case 5 (FIG. 10, IV), showed some dissimilarities.

Fragment ions demonstrating a terminal Hex-HexN-Hex carbohydrate sequence were seen at m/z 187 (219 minus 32), 219, 432 (464 minus 32), 464 and 668 in both spectra. However, in the spectrum of the late-eluting peak of case 5 the fragment ion at m/z 182 was prominent, while this ion was absent in the spectrum of the late-eluting peak of case 4. The fragment ion at m/z 182 is characteristic for type 2 carbohydrate chains, Galβ4GlcNAcβ (41, 42), although it was recently demonstrated that it is only found when the source temperature is set above 280° C. (54).

The fragment ion at m/z 432 (464 minus 32) was also prominent in the spectrum of the saccharide from case 5, as in the spectrum of reference lactoneotetraosylceramide (FIG. 10, II), indicating that methanol is more readily eliminated from Galβ4GlcNAcβ-chains than from Galβ3GlcNAcβchains, most probably from C2-C3.

The saccharide from case 4 gave a strong fragment ion at m/z 228. This ion was also predominant in the spectrum of reference lactotetraosylceramide (FIG. 10, I), and probably originated from the internal GlcNAc, since no ion at m/z 260 was seen.

In conclusion, by gas chromatography and gas chromatography —EI mass spectrometry of permethylated oligosaccharides from the tetraglycosylceramides of case 4 and 5, the results from proton NMR spectroscopy of these fractions were confirmed. The predominant compound of both fractions was identified as globotetraose, while the minor components differed. In the case of the Helicobacter pylori-binding case 4, the minor compound was identified as lactotetraose, while the non-binding case 5 had neolactotetraose.

Frequency of Lactotetraosylceramide Binding Among Helicobacter pylori Isolates

The frequency of expression of the lactotetraosylceramide binding property was estimated by analysing the binding of the 66 Helicobacter pylori isolates listed in Table I to glycosphingolipids on thinlayer chromatograms. For the binding assays the bacteria were grown from stock cultures, and examined for binding of lactotetraosylceramide of human meconium by the chromatogram binding assay. A positive binding indicated a pattern identical to that seen in lane 6 of FIG. 1, B. The strains that failed to bind were re-cultured twice from storage, and re-assayed by the chromatogram binding assay, i. e. no binding to lactotetraosylceramide was detected in three consecutive assays of the strains assigned as non-binding. By these criteria, 9 of the 66 isolates analysed (strain 15, 65, 176, 198, 239, 269, 271, 272 and BH000334 of Table I) were non-binding, while 57 isolates (86%) expressed the lactotetraosylceramide binding capacity.

Discussion

Serologic typing using erythrocytes and saliva demonstrated that the blood group status of case 4 was ALe(a+b−)non-secretor, in agreement with the presence of Helicobacter pylori-binding unsubstituted lactotetraosylceramide in the gastric mucosa of this individual. However, by binding of monoclonal antibodies directed against the Le^{b} determinant a substantial amount of Le^{b}-6 glycosphingolipid was found in the non-acid glycosphingolipid fraction isolated of this individual, and also in the non-acid fractions from the other human stomach specimens. (not shown). This indicates that the expression of Lewis blood group antigens in human gastric mucosa is not correlated with the expression of Lewis antigens on erythrocytes or in saliva, as previously demonstrated for other human tissues, e.g. urothelial tissue (61, 62) and large intestine (47, 63).

The finding that this individual was non-secretor is interesting in view of the increased prevalence of duodenal ulcer among non-secretors (64-66). A recent study (67) demonstrated that non-secretion is not associated with increased susceptibility to infection with Helicobacter pylori. However, the secretor status may determine the outcome of the colonisation, i. e. the increased liability of non-secretors to develop peptic ulcer disease may be due to the presence of the Helicobacter pylori-binding lactotetraosylceramide on the gastric epithelial cells of these individuals.

Under the experimental conditions of the present study, Helicobacter pylori recognised lactotetraosylceramide, while binding to the glycosphingolipids tentatively identified as sulfatide and the GM3 ganglioside in the acid fractions isolated from the epithelial cells of human stomach was non-existent. The binding of Helicobacter pylori to lactotetraosylceramide was not affected by changing the growth conditions, since this binding was obtained both when the bacteria were grown on agar and in broth. Furthermore, binding to lactotetraosylceramide was detected both when the bacteria were grown for 12 h and for 120 h.

Binding to lactotetraosylceramide was also obtained with the babAlA2 mutant strain, where the gene coding for the Le^{b}-binding adhesin had been inactivated (data not shown; ref. 68). Thus, the binding of Helicobacter pylori to the Leb determinant and to lactotetraosylceramide represents two separate binding specificities.

The Le^{b} determinant (Fucα2Galβ3(Fucα4)GlcNAcβ) is based on the type 1 disaccharide unit, which is the terminal part of lactotetraosylceramide. The interaction of Helicobacter pylori with Le^{b} was, however, dependent on fucose with a minimum requirement of the α-fucose in 2-position of the terminal galactose, and with an improved interaction by the substitution of the α-fucose in 4-position of the N-acetylglucosamine (9). In contrast, the binding to lactotetraosylceramide required the unsubstituted carbohydrate chain, since all the substitutions of the basic receptor sequence tested abolished the interaction.

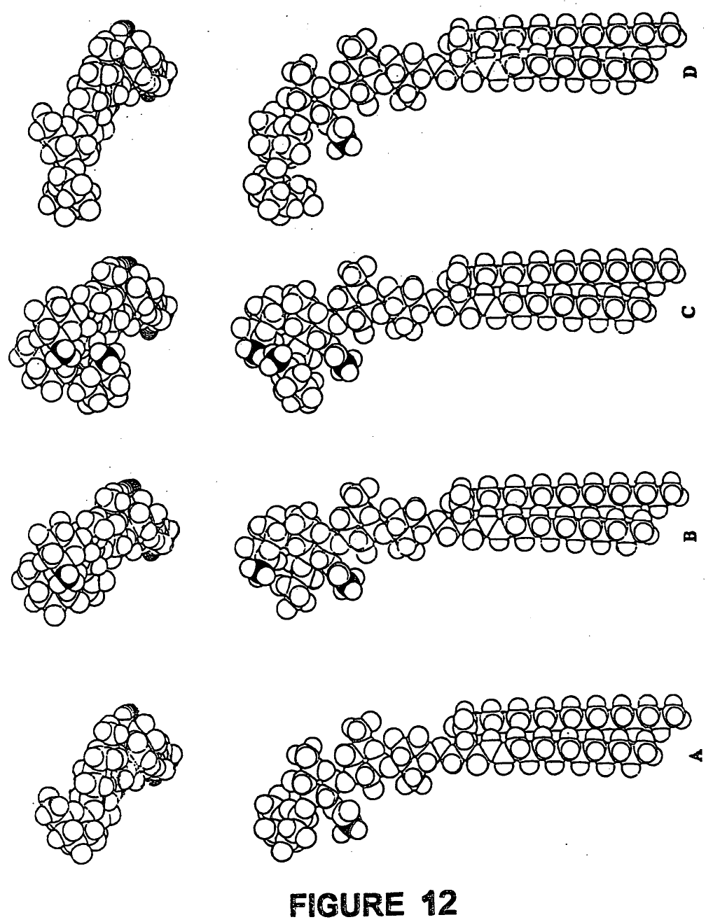

FIG. 12 shows the minimum energy molecular model of lactotetraosylceramide (No. 2 in Table II, FIG. 12 A) in comparison with the Le^{b}-6 glycosphingolipid (No. 6 in Table II, FIG. 12 C) and two other non-binding compounds, namely the Le^{a}-5 glycosphingolipid (No. 5 in Table II, FIG. 12 B) and defucosylated B6 type 1 glycosphingolipid (No. 8 in Table II, FIG. 12 D). The top charts show the same structures viewed from above. The GlcβCer linkage is shown in an extended conformation. The substitutions of the basic lactotetraosylceramide structure in (B)-(D) are dotted, and the methyl carbons of the fucoses and of the acetamido group of GlcNAc are shown in black. In trying to discern the important parts making up the binding epitope of lactotetraosylceramide two observations, the nonbinding of lactotriaosylceramide (No. 1) and of lactotetraosylceramide in which the acetamido moiety has been reduced to an amine (No. 3), indicate that the terminal disaccharide Galβ3GlcNAcβ3 constitutes the epitope. The non-binding of the latter structure (No. 3) further indicates either that an intact acetamido group is essential for binding to occur, or that an altered conformation results since an amine no longer may participate in hydrogen bond interactions with the 2-OH group of the internal Galβ4. A combination of these two effects is also possible. Moreover, extension of the terminal Gal of lactotetraosylceramide by Galα3 (No. 8) or Fucα2 (No. 4), or substitution of the penultimate GlcNAc by Fucα4 (No. 5), yields structures which are inactive, suggesting that the major part of the terminal disaccharide Galβ3GlcNAcβ3 is directly involved in interactions with the adhesin responsible for binding.

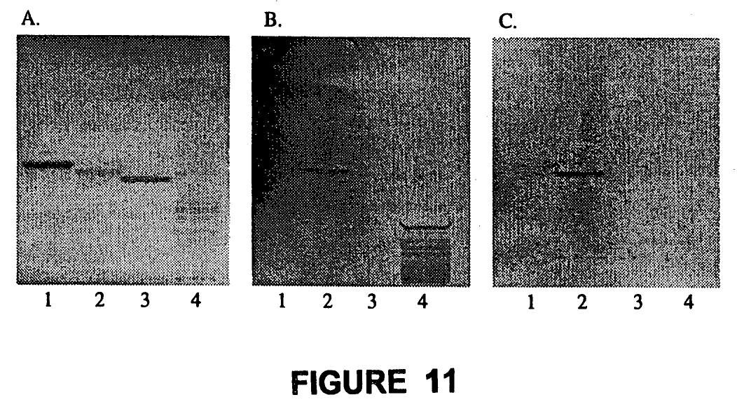

Binding of H. pylori to glycosphingolipids with sialic acid (gangliosides) has been reported (80). However, lactotetraosylceramide is recognised by strains without sialic acid-capacity, as e-g- the strain CCUG 17875, and strains which bind in a sialic dependent manner, as e.g. the strain CCUG 17874 (see FIG. 11). Furthermore, substitution of the terminal Gal of lactotetraosylceramide by an α3-linked sialic acid abolished the binding of H. pylori, as table II, No. 11. Thus, the lactotetraosylceramide binding capacity of H. pylori is not related to the ganglioside recognition.

In the Le^{b} structure the GlcNAcβ3 residue is inaccessible and the penultimate Galβ3 partly so since they are covered by the two fucoses, as seen in the top view (to the left of the page) of FIG. 12 C. Furthermore, since the binding of Helicobacter pylori to Le^{b} is inhibited by the isostructure Le^{y} (9), the GlcNAcβ3 residue of Le^{b} is not essential for binding to this compound. Alignment of the minimum energy structures of the terminal tetrasaccharide part of Le^{b}-6 and Le^{y}-6 shows that the only difference is an approximately 180° turn of the GlcNAcβ3 residue, thus proving the non-requirement of the acetamido moiety of the GlcNAcβ3 residue (or even more likely the whole residue) in the Le^{b} structure, whereas in lactotetraosylceramide the opposite is true. It may be further noted that the angle between the ring plane of the terminal Galβ3 in lactotetraosylceramide and the corresponding plane in the Le^{b} structure is close to 40°, due to the crowdedness caused by the two additional fucose units, affording an additional reason as to why these structures should be regarded as separate receptors for Helicobacter pylori.

FIG. 11 illustrates lactotetraosylceramide recognition both by the sialic acid-binding H. pylori strain CCUG 17874 (B) and the strain CCUG 17875 which is devoid of sialic acid binding capacity (C). The chromatogram in (A) is stained with anisaldehyde. Lane 1=globoside of human erythrocytes (GalNAcβ3Galα4Galβ4Glcβ1Cer), lane 2=lactotetraosylceramid of human meconium (Galβ3GlcNAcβ3Galβ4Glcβ1Cer), lane 3=GM3 ganglioside (NeuAcα3Galβ4Glcβ1Cer), lane 4=gangliosides of human granulocytes. Lactotetraosylceramide (lane 2) is recognised by both strains, while the sialic acid binding capacity of strain CCUG 17874 is demonstrated by the binding to the gangliosides of human granulocytes (lane 4).

Molecular Modelling Experiment

Cross-Binding of Lactotetraosylceramide and Gangliotetraosylceramide Structures

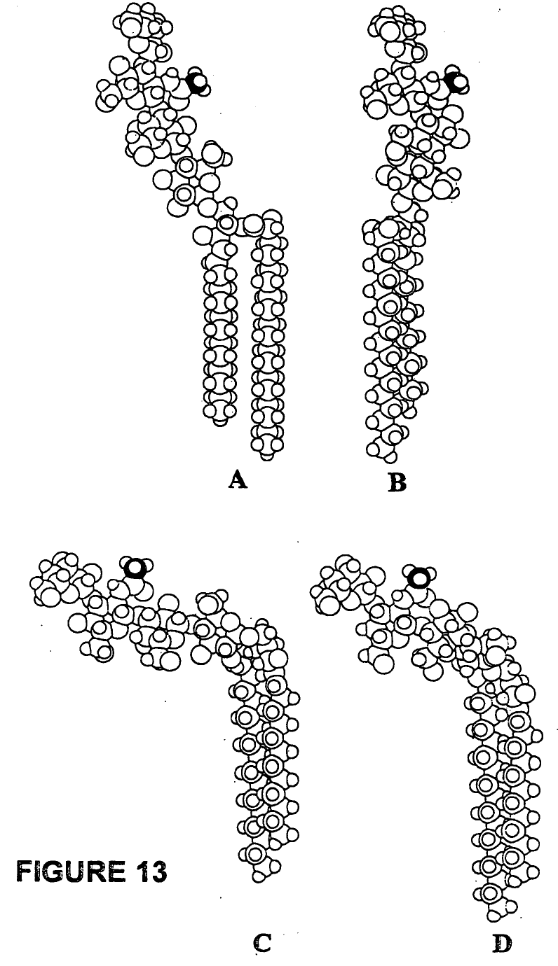

It was recently demonstrated above that H. pylori binds specifically to the terminal disaccharide of lactotetraosylceramide. This has implications for the interpretation of the gangliotetraosylceramide binding epitope since these two structures, where the major difference resides in the linkage between sugar residues two and three, are terminated by the same disaccharide sequence, disregarding the difference at position four of the GalNAc (GlcNAc). Coupled to the observed non-binding of the de-N-acylated species of gangliotriaosylceramide and gangliotetraosylceramide as well as the dramatic increase in affinity on going from the former to the latter glycosphingolipid (16), strongly argues for a case in which the terminal disaccharide also of gangliotetraosylceramide constitutes the binding epitope. Generation and pairwise comparison of the all the likely minimum energy conformers for these lactotetraosylceramide and gangliotetraosylceramide structures show that at least two pairs of conformers result in identical presentation of the respective terminal disaccharide binding epitopes (FIG. 13), suggesting that the same bacterial adhesin may be involved in the binding of the two glycosphingolipids. A difference in the Glcβ1Cer linkage conformations is further indicated by the observation that H. pylori binds to lactotetraosylceramide only when other lipids or bile salts, such as Tween 20 or deoxycholate, are used in the thin-layer chromatogram assay (FIG. 1) whereas gangliotetraosylceramide binding only marginally is affected by such additions. Thus, in the absence of other lipids or bile salts lactotetraosylceramide most likely has a Glcβ1Cer linkage conformation different from the ones shown in FIG. 13 in which the binding epitope is incorrectly presented for binding of H. pylori to occur. Similar effects of other lipids on the conformation of glycosphingolipids have recently been reviewed (73). Moreover, the direct demonstration made above that lactotetraose is able to block binding of H. pylori to gangliotetraosylceramide (FIG. 5) confirms this line of reasoning.

It may thus be concluded that H. pylori binding to the Galβ3GalNAcβ4 epitope of gangliotetraosylceramide actually should be considered as a lactotetraosylceramide specificity with a tolerance for 4-substitution of the internal Gal and for an axial orientation at position four of the GlcNAcβ3 residue.

### Example of an Analogue

Tetrasaccharide Galβ3GlcNAcβ3Galβ4Glc (Isosep, Tullinge, Sweden) and maltoheptaose (Sigma, Saint Louis, USA) were reductively aminated with 4-hexadecylaniline (abbreviation HDA, from Aldrich, Stockholm, Sweden) by cyanoborohydride (Halina Miller-Podraza, to be published later). The products were characterised by mass spectrometry and were confirmed to be Galβ3GlcNAcβ3Galβ4Glc(red) -HDA and maltoheptaose(red) -HDA [where “(red) -” means the amine linkage structure formed by reductive amination from the reducing end glucose of the saccharides and amine group of the hexadecylaniline (HDA)]. The compound Galβ3GlcNAcβ3Galβ4Glc(red) -HDA had similar binding activity with regard to Helicobacter pylori as lactotetraosylceramide glycosphingolipid in TLC-overlay assay described above while the control conjugate maltoheptaose(red) -HDA was totally inactive. The example shows a synthetic derivative of the sequence Galβ3GlcNAc. It also shows that trisaccharide Galβ3GlcNAcβ3Gal is a structure binding to Helicobacter pylori and that glucose at the reducing end is not needed for the binding (reduction destroys the pyranose ring structure of the reducing-end Glc).

TABLE I

| Bacterial strains | Source |
|---|---|
| 4, 15, 17, 48, 51, 54, 56, | Department of Medical Mi- |
| 62, 65, 69, 73, 77, 78, 80, | crobiology, University of |
| 81, 88, 133, 176, 185, 188, | Lund, Sweden |
| 191, 198, 214, 215, 225, 239, | |
| 244, 263, 266, 269, 271, 272, | |
| 275, 287, 306, BH00031, | |
| BH000324, BH000325, BH000331, | |
| BH000332, BH000334 | |
| 002, 005, 032, F6, Ö10, C7050 | Department of Medical Mi- |
| | crobiology and Immunology, |
| | Örebro Medical Centre, Swe- |
| | den |
| 32, 66*, 95*, 915*, 1139*, | Culture Collection Univer- |
| 15816, 17135, 17874*, | sity of Göteborg (CCUG), |
| 17875*, 18430, 18943, 20649 | Sweden |
| 1, 177, 480, 604, 608, 609 | Department of Microbiology, |
| | Medical University of Wro- |
| | claw, Poland |

*From the strains denoted with * cell surface proteins were extracted and used for binding assays, as described above in the “Materials and Methods” section.

TABLE II

| | Trivial | | | | |
|---|---|---|---|---|---|
| No. | name | Glycosphingolipid structure^{a} | Binding^{b} | Source | Ref. |
| 1 | Lacto- | GlcNAcβ3Galβ4Glcβ1Cer | − | Malignant | (c) |
| | tri | | | melanoma | |
| 2 | Lacto- | Galβ3GlcNAcβ3Galβ4Glcβ1Cer | + | Human meconium | (45) |
| | tetra | | | | |
| 3 | | Galβ3GlcNH₂β3Galβ4Glcβ1Cer | − | Human meconium | (d) |
| 4 | H5-1 | Fucα2Galβ3GlcNAcβ3Galβ4Glcβ1Cer | − | Human meconium | (69) |
| 5 | Le^{a}-5 | Galβ3(Fucα4)GlcNAcβ3Galβ4Glcβ1Cer | − | Human small | (70) |
| | | | | intestine | |
| 6 | Le^{b}-6 | Fucα2Galβ3(Fucα4)GlcNAcβ3Galβ4Glcβ1Cer | − | Human small | (71) |
| | | | | intestine | |
| 7 | B6-1 | Galα3(Fucα2)Galβ3GlcNAcβ3Galβ4Glcβ1Cer | − | Monkey intes- | (e) |
| | | | | tine | |
| 8 | | Galα3Galβ3GlcNAcβ3Galβ4Glcβ1Cer | − | Monkey intes- | (f) |
| | | | tine | | |
| 9 | B7-1 | Galα3(Fucα2)Galβ(Fucα4)3GlcNAcβ3Galβ4Glcβ1Cer | − | Monkey intes- | (e) |
| | | | | tine | |
| 10 | A6-1 | GalNAcα3(Fucα2)Galβ3GlcNAcβ3Galβ4Glcβ1Cer | − | Human meconium | (69) |
| 11 | | NeuGcα3Galβ3GlcNAcβ3Galβ4Glcβ1Cer | − | Rabbit thymus | (81) |

^{a}The GalβGlcNAc parts have been underlined.
^{b}+ marks a significant darkening on the autoradiogram when 2 μg was applied on the thin-layer plate, while - marks no darkening.
(c) B. E. Samuelsson and K. -A. Karisson, unpublished results.
(d) Glycosphingolipid No. 3 was produced from GalβGlcNAcβ3Galβ4Glcβ31Cer from human meconium (No. 2) by treatment with anhydrous hydrazine (to be reported separately).
(e) N. Strömberg and K. -A. Karlsson, unpublished results.
(f) Glycosphingolipid No. 8 was generated from Galα3(Fucα2)Galβ3GlcNAcβ3Galβ4Glcβ1Cer from monkey intestine (No. 7) by incubation in 0.05 M HCl at 80° C. for 2 h.

TABLE III

| | | | Non-acid | Acid |
|---|---|---|---|---|
| Case | Blood | | glycosphingo- | glycosphingo- |
| No. | group | Tissue | lipids | lipids |
| 1 | ORh− | Mucosal | 7.0^{a} (11.9)^{b} | 8.5^{a} (14.4)^{b} |
| | | cells | 2.7 (6.0) | 22.0 (48.8) |
| | | Non-mucosal | | |
| | | residue | | |
| 2 | ARh+ | Mucosal | 3.6 (18.0) | 10.7 (53.5) |
| | | cells | | |
| 3 | ARh+ | Mucosal | 6.4 (14.5) | 2.9 (6.6) |
| | | cells | | |
| 4 | ARh+ | Mucosal | 6.0 (24.0) | 4.8 (19.2) |
| | | cells | | |
| 5 | ARh+ | Mucosal | 23.0 (38.0) | .5 (9.2) |
| | | cells | | |
| 6 | ARh− | Mucosal | 4.9 (18.1) | 8.2 (30.4) |
| | | cells | | |
| 7 | Unknown | Mucosal | 2.5 (15.6) | 7.5 (46.8) |
| | | cells | 4.3 (8.7) | 7.6 (15.5) |
| | | Non-mucosal | | |
| | | residue | | |

### References

1. Beachey, E. H. (1981) J. Infect. Dis. 143, 325-345

2. Blaser, M. J. (1990) J. Infect. Dis. 161, 626-633

3. Blaser, M. J. (1992) Eur. J. Gastroenterol. Hepatol. 4 (suppl 1), 17-19

4. Mégraud, F. and Lamouliatte, H. (1992) Dig. Dis. Sci. 37, 769-772

5. Dooley, C. P. (1993) Curr. Op. Gastroenterol. 9, 112-117

6. Eurogast Study Group (1993) Lancet 341, 1359-1362

7. Solnick, J. V. and Tompkins, L. S. (1993) Infect. Agents Dis. 1, 294-309

8. Bode, G., Malfertheiner, P. and Ditschuneit, H. (1988) Scand. J. Gastroenterol. 23 (suppl 142), 25-39

9. Falk, P., Roth, K. A., Borén, T., Westblom, T. U., Gordon, J. I. and Normark, S. (1993) Proc. Natl. Acad. Sci. 90, 2035-2039

10. Lingwood, C. A., Huesca, M. and Kuksis, A. (1992) Infect. Immun. 60, 2470-2474

11. Gold, B., Huesca, M., Sheran, P. and Lingwood, C. A. (1993) INFECT. IMMUN. 61, 2632-2638

12. Borén, T., Falk, P., Roth, K. A., Larson, G. and Normark, S. (1993) Science 262, 1892-1895

13. Ascencio, F., Fransson, L. Å. and Wadström, T. (1993) J. Med. Microbiol. 38, 240-244

14. Saitoh, T., Natomi, H., Zhao, W., Okuzumi, K., Sugano, K., Iwamori, M. and Nagai, Y. (1991) FEBS Lett. 282, 385-387

15. Kamisago, S., Iwamori, M., Tai, T., Mitamura, K., Yazaki, Y. and Sugano, K. (1996) Infect. Immun. 64, 624-628

16. Ångström, J., Teneberg, S., Abul Milh, M., Larsson, T., Leonardsson, I., Olsson, B.-M., Ölwegård Halvarsson, M., Danielsson, D., Näslund, I., Ljungh, Å., Wadström, T., Karlsson, K.-A. (1998) Glycobiology 8, 297-309

17. Miller-Podraza, H., Bergström, J., Abul Milh, M. and Karlsson, K.-A. (1997) Glycoconj. J. 14, 467-472

18. Miller-Podraza, H., Abul Milh, M., Teneberg, S. and Karlsson, K.-A. (1997) Infect. Immun. 65, 2480-2482

19. Soltesz, V., Schalén, C. and Mårdh, P.-A. (1988) in Campylobacter IV. Proceedings of fourth International Workshop on Campylobacter infection (Kaijser, B. and Falsen, E., eds.) pp. 433-436, Goterna, Kungälv, Sweden

20. Lelwala-Guruge, J., Nilsson, I., Ljungh, Å. and Wadström, T. (1992) Scand. J. Infect. Dis. 24, 457-465

21. Aggarwal, B. B., Eessalu, T. E. and Hass, P. E. (1985) Nature 318, 665-667

22. Waldi, D. (1962) in Dünnschicht-Chromatographie (Stahl, E., ed.) pp. 496-515. Springer-Verlag, Berlin

23. Svennerholm, L. (1963) J. Neurochem. 10, 613-623

24. Hansson, G. C., Karlsson, K.-A., Larson, G., Strömberg, N. and Thurin, J. (1.985) Anal. Biochem. 146, 158-163

25. Karlsson, K.-A. (1987) Meth. Enzymol. 138, 212-220

26. Samuelsson, B. E., Pimlott, W. and Karlsson K.-A. (1990) Meth. Enzymol. 193, 623-46

27. Falk, K.-E., Karlsson, K.-A. and Samuelsson B. E. (1979) Arch. Biochem. Biophys. 192, 164-176

28. Falk, K.-E., Karlsson, K.-A. and Samuelsson B. E. (1979) Arch. Biochem. Biophys. 192, 177-190

29. Falk, K.-E., Karlsson, K.-A. and Samuelsson B. E. (1979) Arch. Biochem. Biophys. 192, 191-202

30. Koerner Jr, T. A. W., Prestegard, J. H., Demou, P. C. and Yu, R. K. (1983) Biochemistry 22, 2676-2687

31. Yang, H. and Hakomori, S.-i. (1971) J. Biol. Chem. 246, 1192-1200

32. Stellner, K., Saito, H. and Hakomori, S.-i. (1973) Arch. Biochem. Biophys. 155, 464-472

33. Larson, G., Karlsson, H., Hansson, G. C. and Pimlott, W. (1987) Carbohydr. Res. 161, 281-290

34. Breimer, M., Hansson, G. C., Karlsson, K.-A., Larson, G., Leffler, H., Pascher, I., Pimlott, W. and Samuelsson, B. E. (1980) in Advances in Mass Spectrometry (Quayle, A., ed.) Vol. 8, pp. 1097-1108, Heyden & Son, London

35. Mayo, S. L., Olafson, B. D. and Goddard III, W. A. (1990) J. Phys. Chem. 94, 8897-8909

36. Rappé, A. K. and Goddard III, W. A. (1990) J. Phys. Chem. 95, 3358-3363

37. Hansson, G. C., Li, Y.-T. and Karlsson, H. (1989) Biochemistry 28, 6672-6678

38. Hakomori, S.-i. (1983) in Sphingolipid Biochemistry (Kanfer, J. N. and Hakomori, S.-i., eds.) Vol-3, pp. 1-165, Plenum Press, N.Y.

39. Ledeen, R. W. and Yu, R. K. (1978) Res. Methods Neurochem. 4, 371-410

40. Karlsson, H., Karlsson, N. and Hansson, G. C. (1994) Molec. Biotechnol. 1, 165-180

41. Karlsson, K.-A. (1976) in Glycolipid Methodology (Witting, L. A., ed.) pp. 97-122, Am. Oil. Soc., Champaign, Ill

42. Karlsson, K.-A. (1978) Progr. Chem. Fats Other Lipids 16, 207-230

43. Holmes, E. H. and Levery, S. B. (1989) Arch. Biochem. Biophys. 274, 14-25

44. Kannagi, R., Levery, S. B. and Hakomori, S.-i. (1984) J. Biol. Chem. 259, 8444-8451

45. Karlsson, K.-A. and Larson, G. (1979) J. Biol. Chem. 254, 9311-9316

46. Björk, S., Breimer, M. E., Hansson, G. C., Karlsson, K.-A. and Leffler, H. (1987) J. Biol. Chem. 262, 6758-6765

47. Holgersson, J., Jovall, P.-Å. and Breimer, M. E. (1991) J. Biochem.. 110, 120-131

48. Breimer, M. E., Cedergren, B., Karlsson, K.-A., Nilsson, K. and Samuelsson, B. E. (1980) FEBS Lett. 118, 209-211

49. Marcus, D. M., Naiki, M. and Kundu, S. K. (1976) Proc. Natl. Acad. Sci. 73, 3263-3267

50. Warren, J. R. and Marshall, B. J. (1983) Lancet i, 1273-1275

51. Rauws, E. A. J. and Tytgat, G. N. J. (1989) in Campylobacter pylori. pp. 49-88, W C den Ouden BV, Amsterdam

52. Gasa, S., Mitsuyama, T. and Makita, A. (1983) J. Lipid Res. 24, 174-182

53. Clausen, H., Levery, S. B., Kannagi, R. and Hakomori, S.-i. (1986) J. Biol. Chem. 261, 1380-1387

54. Bouhours, D., Bouhours, J.-F., Larson, G., Karlsson, H., Pimlott, W. and Hansson, G. C. (1990) Arch. Biochem. Biophys. 282, 141-146

55. Natomi, H., Saitoh, T., Sugano, K., Iwamori, M., Fukayama, M. and Nagai, Y. (1993) Lipids 28, 737-742

56. Dohi, T., Ohta, S., Hanai, N., Yamaguchi, K. and Oshima, M. (1990) J. Biol. Chem. 265, 7880-7885

57. Dohi, T., Hanai, N., Yamaguchi, K. and Oshima, M. (1991) J. Biol. Chem. 266, 24038-24043

58. Hattori, H., Uemura, K.-I. and Taketomi, T. (1981) Biochim. Biophys. Acta 666, 361-369

59. Blasco, E., Gutierrez-Hoyos, A., Lojendio, M., Cosme, A. and Arenas, J. I. (1991) Eur. J. Cancer 27, 501-503

60. Henry, S. M., Samuelsson, B. E. and Oriol, R. (1994) Glycoconj. J. 11, 600-607

61. Πrntoft, T. F., Wolf, H., Clausen, H., Hakomori, S.-i. and Dabelsteen, E. (1987) J. Urol. 138, 171-176

62. Πrntoft, T. F., Holmes, E., Johnson, P., Hakomori, S.-i. and Clausen, H. (1991) Blood 77, 1389-1396

63. Holgersson, J., Strömberg, N. and Breimer, M. E. (1988) Biochimime 70, 1565-1574

64. Clark, C. A., Wyn Edwards, J., Haddock, D. R. W., Howel-Evans, A. W., McConnell, R. B. and Sheppard, P. M. (1956) BMJ 2, 725-731

65. Rotter, J. I. (1983) in Principles and Practice of Medical Genetics (Emery, A. E. H. and Riomoin, D. L., eds.) pp. 863-878, Churchill Livingstone, N.Y.

66. Mentis, A., Blackwell, C. C., Weir, D. M., Spiliadis, C., Dailianas, A. and Skandalis, N. (1991) Epidemiol. Infect. 106, 221-229

67. Dickey, W., Collins, J. S. A., Watson, R. P. G., Sloan, J. M. and Porter, K. G. (1993) Gut 43, 351-353

68. Falk, P. G., Bry, L., Holgersson, J. and Gordon, J. I. (1995) Proc. Natl. Acad. Sci. USA 92, 1515-1519

69. Karlsson, K.-A. and Larson, G. (1981) J. Biol. Chem. 256, 3512-3524

70. Smith, E. L., McKibbin, J. M., Karlsson, K.-A., Pascher, I., Samuelsson, B. E., Li, Y.-T. and Li, S.-C. (1975) J. Biol. Chem. 250, 6059-6064

71. McKibbin, J. M., Spencer, W. A., Smith, E. L., Månsson, J.-E., Karlsson, K.-A., Samuelsson, B. E., Li, Y.-T. and Li, S.-C. (1975) J. Biol. Chem. 257, 755-760

72. Pingren, H., Hu, J. and Macher, B. A. (1993) Arch. Biochem. Biophys. 305, 350-361

73. Maggio, B. (1994) Prog. Biophys. Molec. Biol., 62, 55-117

74. Pakodi, F., Abdel-Salam, O. M. E., Debreceni, A., and Mózsik, G. (2000) J. Physiol. (Paris), 94, 139-152

75. Farsak, B., Yildirir, A., Akyön, Y., Pinar. A., Öc, M., B\ddot{k}e, E., Kes, S., Tokgözoglu, L. (2000) J. Clin. Microbiol. 38, 4408-11

76. Nilsson, H-O., Taneera, J., Castedal, M., Glatz, E., Olsson, R., and Wadström, T. (2000) J. Clin. Microbiol. 38, 1072-1076

77. Avenaud, P., Marais, A., Monteiro, L., Le Bail, B., Biolac Sace, P., Balabaud, C., and Mégraud, F. (2000) Cancer 89, 1431-1439

78. Rebora, R., Drago, F., and Parodi, A. (1995) Dermatology 191, 6-8

79. Kerr, J. R., Al-Khattaf, A., Barson, A. J., and Burnie, J. P. (2000) Arch. Dis. Child. 83, 429-434

80. Miller-Podraza, H., Abul Milh, M., Teneberg, S., and Karlsson K.-A. (1997) INFECT. IMMUN. 65, 2480-2482

81. Iwamori, M., Iwamoto, M., Hayashi, K., Kigushi, K. and Nagai, Y. (1985) J. Biochem. (Tokyo) 98, 1561-1569

1. A Helicobacter pylori-binding substance comprising at least one compound having Formula 1: 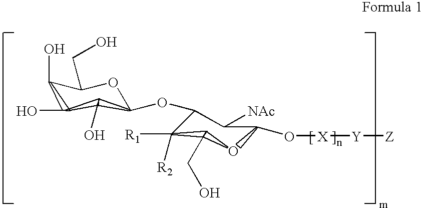
wherein:
R₁ is H or OH and R₂ is H or OH, under the provision that R₁ is H when R₂ is OH and R₁ is OH when R₂ is H;
X is a monosaccharide or oligosaccharide residue;
Y is nothing, a spacer group or a terminal conjugate;
Z is an oligovalent or a polyvalent carrier or —H;
n is 0 or 1;
m is an integer ≧1,
or an analogue or derivative thereof having the same or better binding activity as the compound having formula I with regard to Helicobacter pylori.

2. A Helicobacter pylori-binding substance comprising at least one compound having Formula 2: 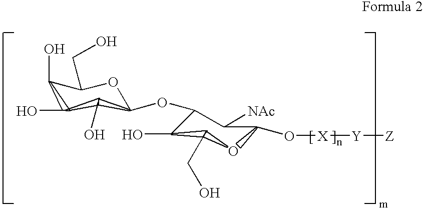
wherein:
X is a monosaccharide or oligosaccharide residue;
Y is nothing, a spacer group or a terminal conjugate;
Z is an oligovalent or a polyvalent carrier or —H;
n is 0 or 1;
m is an integer ≧1,
or an analogue or derivative thereof having the same or better binding activity as the compound having formula I with regard to Helicobacter pylori.

3. A Helicobacter pylori-binding substance comprising at least one compound having Formula 3: 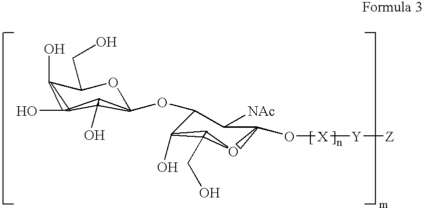
wherein:
X is a monosaccharide or oligosaccharide residue;
Y is nothing, a spacer group or a terminal conjugate;
Z is an oligovalent or a polyvalent carrier or —H;
n is 0 or 1;
m is an integer ≧1,
or an analogue or derivative thereof having the same or better binding activity as the compound having formula I with regard to Helicobacter pylori.

4. A Helicobacter pylori-binding substance comprising Galβ3GlcNAc, or an analogue or derivative thereof having the same or better binding activity as Galβ3GlcNAc with regard to Helicobacter pylori.

5. A Helicobacter pylori-binding substance according to claim 4, wherein said Galβ3GlcNAc, or analogue or derivative thereof, is at a terminal non-reducing end of said substance.

6. A Helicobacter pylori-binding substance according to claim 4, consisting of Galβ3GlcNAc or an analogue or derivative thereof having the same or better binding activity as Galβ3GlcNAc with regard to Helicobacter pylori.

7. A Helicobacter pylori-binding substance comprising Galβ3GalNAc, or an analogue or derivative thereof having the same or better binding activity as Galβ3GlcNAc with regard to Helicobacter pylori.

8. A Helicobacter pylori-binding substance according to claim 7, wherein said Galβ3GalNAc, or analogue or derivative thereof, is at a terminal non-reducing end of said substance.

9. A Helicobacter pylori-binding substance according to claim 7, consisting of Galβ3GalNAc or an analogue or derivative thereof having the same or better binding activity as Galβ3GlcNAc with regard to Helicobacter pylori.

10. A Helicobacter pylori-binding substance according to claim 4 or 5, comprising lactotetraose.

11. A Helicobacter pylori-binding substance according to claim 4 or 5, consisting of lactotetraose.

12. A Helicobacter pylori-binding substance according to claim 4 or 5, comprising lactotetraosylceramide (Galβ3GlcNAcβ3Galβ4Glcβ1Cer).

13. A Helicobacter pylori-binding substance according to claim 4 or 5, consisting of lactotetraosylceramide (Galβ3GlcNAcβ3Galβ4Glcβ1Cer).

14. A Helicobacter pylori-binding substance according to claim 7 or 8 comprising gangliotetraosylceramide (Galβ3GalNAcβ4Galβ4Glcβ1Cer).

15. A Helicobacter pylori-binding substance consisting of a carrier to which one or more of the substances according to the claims 1-14 has/have been attached.

16. A Helicobacter pylori-binding substance consisting of a micelle comprising one or more of the substances according to the claims 1-15.

17. A Helicobacter pylori-binding substance comprising one or more of the substances according to the claims 1-16 conjugated to a polysaccharide.

18. A Helicobacter pylori-binding substance according to claim 17, wherein said polysaccharide is a polylactosamine chain or a conjugate thereof.

19. A Helicobacter pylori-binding substance according to any one of the claims 1-14, said substance being a glycolipid.

20. A Helicobacter pylori-binding substance according to any one of the claims 1-14, said substance being a glycoprotein or a neoglycoprotein.

21. A Helicobacter pylori-binding substance according to any one of the claims 1-14, said substance being an oligomeric molecule comprising at least two oligosaccharide chains.

22. A Helicobacter pylori-binding substance according to any one of the claims 1-14, said substance being an oligomeric molecule comprising at least three oligosaccharide chains.

23. A Helicobacter pylori-binding substance comprising one or more of the substances according to the claims 1-22 covalently conjugated with an antibiotic effective against Helicobacter pylori.

24. A pharmaceutical composition comprising a substance according to any one of the claims 1-23.

25. A pharmaceutical composition according to claim 24, for treatment of a condition due to the presence of Helicobacter pylori.

26. A pharmaceutical composition according to claim 24 or claim 25, for treatment of a condition due to the presence of Helicobacter pylori in the gastrointestinal tract of a patient.

27. A pharmaceutical composition according to any one of the claims 24-26, for treatment of chronic superficial gastritis.

28. A pharmaceutical composition according to any one of the claims 24-26, for treatment of duodenal ulcer.

29. A pharmaceutical composition according to any one of the claims 24-26 for treatment of gastric ulcer.

30. A pharmaceutical composition according to any one of the claims 24-26, for treatment of gastric adenocarcinoma.

31. A pharmaceutical composition according to any one of the claims 24-26, for treatment of non-Hodgkin lymphoma of human stomach.

32. A pharmaceutical composition according to claim 24 or 25, for treatment of a liver disease.

33. A pharmaceutical composition according to claim 24 or 25, for treatment of a heart disease.

34. A pharmaceutical composition according to any one of the claims 24-26, for treatment of sudden infant death syndrome.

35. Use of a substance according to any one of the claims 1-23 for the production of a pharmaceutical composition for treatment of a condition due to the presence of Helicobacter pylori.

36. Use of a substance according to any one of the claims 1-23 for the production of a pharmaceutical composition for treatment of a condition due to the presence of Helicobacter pylori in the gastrointestinal tract of a patient.

37. Use according to claim 35 or 36, wherein said pharmaceutical composition is intended for treatment of chronic superficial gastritis.

38. Use according to claim 35 or 36, wherein said pharmaceutical composition is intended for treatment of duodenal ulcer.

39. Use according to claim 35 or 36, wherein said pharmaceutical composition is intended for treatment of gastric ulcer.

40. Use according to claim 35 or 36, wherein said pharmaceutical composition is intended for treatment of gastric adenocarcinoma.

41. Use according to claim 35 or 36, wherein said pharmaceutical composition is intended for treatment of non-Hodgkin lymphoma of human stomach.

42. Use according to claim 35, wherein said pharmaceutical composition is intended for treatment of a liver disease.

43. Use according to claim 35, wherein said pharmaceutical composition is intended for treatment of a heart disease.

44. Use according to claim 35 or 36, wherein said pharmaceutical composition is intended for treatment of sudden infant death syndrome.

45. Use of a substance according to any one of the claims 1-23 for inhibition of the binding of Helicobacter pylori.

46. Use of a substance according to claim 45 for inhibition of the binding of Helicobacter pylori for non-medical purposes.

47. Use according to claim 46 in an assay system.

48. Use according to claim 47, wherein said assay system is used for the identification of other Helicobacter pylori-binding substances.

49. Use of a substance according to any one of the claims 1-23 as a lead compound in the identification of other Helicobacter pylori-binding substances.

50. Food-stuff comprising a substance according to any one of the claims 1-23.

51. A nutritional additive comprising a substance according to any one of the claims 1-23.

52. Food-stuff according to claim 50 or a nutritional additive according to claim 51, wherein said substance is Galβ3GlcNAcβ3Galβ4Glc.

53. Food-stuff according to claim 52, in the form of an infant formula food.

54. Food-stuff according to claim 53, wherein said Galβ3GlcNAcβ3Galβ4Glc is intended for use with a concentration of Galβ3GlcNAcβ3Galβ4Glc of 0.1-0.5 g/l.

55. Food stuff according to claim 54, wherein said concentration is 0.05-5 g/l.

56. Use of a food-stuff or a nutritional additive according to any one of the claims 50-55 for the inhibition of the binding of Helicobacter pylori.

57. A method for treatment of a condition due to the presence of Helicobacter pylori in a patient, wherein a pharmaceutically effective amount of a substance according to any one of the claims 1-23 is administered to the patient.

58. A method for treatment of a condition due to the presence of Helicobacter pylori in a patient, wherein a food stuff according to any one of the claims 50-55 is administered to the patient.

59. A method according to claim 57 or 58, wherein said condition is due to the presence of Helicobacter pylori in the gastrointestinal tract of said patient.

60. A method according to claim 57 or 58, for treatment of chronic superficial gastritis.

61. A method according to claim 57 or 58, for treatment of duodenal ulcer.

62. A method according to claim 57 or 58, for treatment of gastric ulcer.

63. A method according to claim 57 or 58, for treatment of gastric adenocarcinoma.

64. A method according to claim 57 or 58, for treatment of non-Hodgkin lymphoma of human stomach.

65. A method according to claim 57 or 58, for treatment of a liver disease.

66. A method according to claim 57 or 58, for treatment of a heart disease.

67. A method according to claim 57 or 58, for treatment of sudden infant death syndrome.

68. Use of a substance according to any one of the claims 2, 4-6, or 10-13 for the identification of bacterial adhesin.

69. Use of a substance according to any one of the claims 1-23 or a substance identified according to claim 68 for the production of a vaccine against Helicobacter pylori.

70. A vaccine against Helicobacter pylori infections produced by use of a substance according to any one of the claims 1-23, or a substance identified according to claim 68.

71. Use of a substance according to any one of the claims 1-23 in the diagnosis of a condition due to a Helicobacter pylori infection.

72. Use of a substance according to any one of the claims 1-23 for typing of Helicobacter pylori.
